SEQUENCE LISTING (41 sequences)

SEQ ID NO: 1 - protein, 58 aa, synthetic construct [sequence omitted]

SEQ ID NO: 2 - protein, 58 aa, synthetic construct
VDNKFNKEXXXAXXEIXXLPNLNXXQXXAFIXSLXDDPSQSANLLAEAKKLNDAQAPK

SEQ ID NO: 3 - protein, 58 aa, synthetic construct
VDNKFNKESSSASSEISSLPNLNSSQSSAFISSLSDDPSQSANLLAEAKKLNDAQAPK

SEQ ID NO: 4 - protein, 76 aa, synthetic construct [sequence omitted]

SEQ ID NO: 5 - protein, 76 aa, synthetic construct
MXIXVXTLTGKTITLEVEPSDTIENVKAKIQDKEGIPPDQQRLIFAGKQLEDGRTLSDYNIXXXXXLHLVLRLRGG

SEQ ID NO: 6 - protein, 76 aa, synthetic construct
MSISVSTLTGKTITLEVEPSDTIENVKAKIQDKEGIPPDQQRLIFAGKQLEDGRTLSDYNISSSSSLHLVLRLRGG

SEQ ID NO: 7 - protein, 162 aa, synthetic construct
MQIFVKTLTGKTITLEVEPSDTIENVKAKIQDKEGIPPDQQRLIFAGKQLEDGRTLSDYNIQKESTLHLVLRLRGGSGGGSGGGIGMQIFVKTLTGKTITLEVEPSDTIENVKAKIQDKEGIPPDQQRLIFAGKQLEDGRTLSDYNIQKESTLHLVLRLRGG

SEQ ID NO: 8 - protein, 162 aa, synthetic construct
MXIXVXTLTGKTITLEVEPSDTIENVKAKIQDKEGIPPDQQRLIFAGKQLEDGRTLSDYNIXXXXXLHLVLRLRGGSGGGSGGGIGMQIFVXTXTGKTITLEVEPSDTIENVKAKIQDKEGIPPDQQRLIFAGKQLEDGRTLSDYNIXXXXXLHLVLRLRGG

SEQ ID NO: 9 - protein, 162 aa, synthetic construct
MSISVSTLTGKTITLEVEPSDTIENVKAKIQDKEGIPPDQQRLIFAGKQLEDGRTLSDYNISSSSSLHLVLRLRGGSGGGSGGGIGMQIFVSTSTGKTITLEVEPSDTIENVKAKIQDKEGIPPDQQRLIFAGKQLEDGRTLSDYNISSSSSLHLVLRLRGG

SEQ ID NO: 10 - protein, 175 aa, synthetic construct
MGKITFYEDRGFQGHCYECSSDCPNLQPYFSRCNSIRVDSGCWMLYERPNYQGHQYFLRRGDYPDYQQWMGFNDSIRSCRLIPQHTGTFRMRIYERDDFRGQMSEITDDCPSLQDRFHLTEVHSLNVLEGSWVLYEMPSYRGRQYLLRPGEYRRYLDWGAMNAKVGSLRRVMDFY

SEQ ID NO: 11 - protein, 175 aa, synthetic construct
MGXIXFXEDRGFQGHXYXCXSDCPNLQPYFSRCNSIXVXSGCWMLYERPNYQGHQYFLRRGDYPDYQQWMGFNDSIRSCRLIPQHTGTFRMRIYERDDFRGQMSEITDDCPSLQDRFHLTEVHSLNVLEGSWVLYEMPSYRGRQYLLRPGEYRRYLDWGAMNAKVGSLRRVMDFY

SEQ ID NO: 12 - protein, 175 aa, synthetic construct
MGSISFSEDRGFQGHSYSCSSDCPNLQPYFSRCNSISVSSGCWMLYERPNYQGHQYFLRRGDYPDYQQWMGFNDSIRSCRLIPQHTGTFRMRIYERDDFRGQMSEITDDCPSLQDRFHLTEVHSLNVLEGSWVLYEMPSYRGRQYLLRPGEYRRYLDWGAMNAKVGSLRRVMDFY

SEQ ID NO: 13 - protein, 178 aa, synthetic construct
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 14 - protein, 178 aa, synthetic construct
QDSTSDLIPAPPLSKVPLQQNFQDNQFQGKWYVVGXAGNXXLREDKDPXKMXATIYELKEDKSYNVTXVXFXXKKCXYXIXTFVPGCQPGEFTLGXIKSXPGXTSXLVRVVSTNYNQHAMVFFKXVXQNREXFXITLYGRTKELTSELKENFIRFSKSLGLPENHIVFPVPIDQCIDG

SEQ ID NO: 15 - protein, 178 aa, synthetic construct
QDSTSDLIPAPPLSKVPLQQNFQDNQFQGKWYVVGSAGNSSLREDKDPSKMSATIYELKEDKSYNVTSVSFSSKKCSYSISTFVPGCQPGEFTLGSIKSSPGSTSSLVRVVSTNYNQHAMVFFKSVSQNRESFSITLYGRTKELTSELKENFIRFSKSLGLPENHIVFPVPIDQCIDG

SEQ ID NO: 16 - protein, 201 aa, synthetic construct
MELWGAYLLLCLFSLLTQVTTEPPTQKPKKIVNAKKDVVNTKMFEELKSRLDTLAQEVALLKEQQALQTVCLKGTKVHMKCFLAFTQTKTFHEASEDCISRGGTLGTPQTGSENDALYEYLRQSVGNEAEIWLGLNDMAAEGTWVDMTGARIAYKNWETEITAQPDGGKTENCAVLSGAANGWFDKRCRDQLPYICQFGIV

SEQ ID NO: 17 - protein, 201 aa, synthetic construct
MELWGAYLLLCLFSLLTQVTTEPPTQKPKKIVNAKKDVVNTKMFEELKSRLDTLAQEVALLKEQQALQTVCLKGTKVHMKCFLAFTQTKTFHEASEDCISRGGTLGTPQTGSENDALYEYLRQSVGNEAEIWLGLNXXXXXXXWVDMTGARIAYKNWETEITAQPDXXXXENCAVLSGAANGWFDKRCRDQLPYICQFGIV

SEQ ID NO: 18 - protein, 201 aa, synthetic construct
MELWGAYLLLCLFSLLTQVTTEPPTQKPKKIVNAKKDVVNTKMFEELKSRLDTLAQEVALLKEQQALQTVCLKGTKVHMKCFLAFTQTKTFHEASEDCISRGGTLGTPQTGSENDALYEYLRQSVGNEAEIWLGLNSSSSSSSWVDMTGARIAYKNWETEITAQPDSSSSENCAVLSGAANGWFDKRCRDQLPYICQFGIV

SEQ ID NO: 19 - protein, 91 aa, synthetic construct
DLGKKLLEAARAGQDDEVRILMANGADVNABXXGXTPLHLAAXXGHLEIVEVLLKZGADVNAXYDKFGKTAFDISIDNGNEDLAEILQKLN

SEQ ID NO: 20 - protein, 90 aa, synthetic construct
DLGKKLLEAARAGQDDEVRILMANGADVNABXXGXTPLHLAAXXGHLEIVEVLLKZGADVNAXDKFGKTAFDISIDNGNEDLAEILQKLN

SEQ ID NO: 21 - protein, 90 aa, synthetic construct
DLGKKLLEAARAGQDDEVRILMANGADVNADSSGSTPLHLAASSGHLEIVEVLLKNGADVNASDKFGKTAFDISIDNGNEDLAEILQKLN

SEQ ID NO: 22 - protein, 63 aa, synthetic construct
GVTLFVALYDYEARTEDDLSFHKGEKFQILNSSEGDWWEARSLTTGETGYIPSNYVAPVDSIQ

SEQ ID NO: 23 - protein, 63 aa, synthetic construct
GVTLFVALYDYXXXXXXDLSFHKGEKFQILXXXXGDWWEARSLTTGETGYIPSNYVAPVDSIQ

SEQ ID NO: 24 - protein, 63 aa, synthetic construct
GVTLFVALYDYSSSSSSDLSFHKGEKFQILSSSSGDWWEARSLTTGETGYIPSNYVAPVDSIQ

SEQ ID NO: 25 - protein, 111 aa, synthetic construct
VYPKKTHWTAEITPNLHGTEVVVAGWVWELRDIGRVKFVVVRDREGGAFVQVTLKAGKTPDHLFKVFAELSREDVVVIKGIVEASKIAKSGVEIFPSEIWILNKAKPLPID

SEQ ID NO: 26 - protein, 111 aa, synthetic construct
VYPKKTHWTAEITPNLHGTEVVVAGWVXXLXDXGXXKXVXVXDREGGAXVXVXLXAGKTPDHLFKVFAELSREDVVVIKGIVEASKXXXXGVEIFPSEIWILNKAKPLPID

SEQ ID NO: 27 - protein, 111 aa, synthetic construct
VYPKKTHWTAEITPNLHGTEVVVAGWVSSLSDSGSSKSVSVSDREGGASVSVSLSAGKTPDHLFKVFAELSREDVVVIKGIVEASKSSSSGVEIFPSEIWILNKAKPLPID

SEQ ID NO: 28 - protein, 114 aa, synthetic construct
IEEIQKQIAAIQKQIAAIQKQIYRMTGGSGGGSGGGSGGGSGMSIEEIQKQIAAIQKQIAAIQKQIYRMTTGGSGGGSGGGSGGGSGMSIEEIQKQIAAIQKQIAAIQKQIYRM

SEQ ID NO: 29 - protein, 114 aa, synthetic construct
IEEIQKXIAXIQEXIAXIQKXIYXMTGGSGGGSGGGSGGGSGMSIEEIQKQIAAIQKQIAAIQKQIYRMTTGGSGGGSGGGSGGGSGMSIEEIQKQIXAIXEQIXAIXKQIXAM

SEQ ID NO: 30 - protein, 114 aa, synthetic construct
IEEIQKQIAAIQKQIAAIQKQIYAMTGGSGGGSGGGSGGGSGMSIEEIQKQIAAIQKQIAAIQKQIYRMTTGGSGGGSGGGSGGGSGMSIEEIQXQIXXIQXQIXXIQXQIXXM

SEQ ID NO: 31 - protein, 93 aa, synthetic construct
IQQIQKXIAXIQEXIYXMTGGSGGGSGGGSGGGSGMDIQQIQKQIAAIQKQIYAMTTGGSGGGSGGGSGGGSGMDIQQIQKQIXAIXEQIXAM

SEQ ID NO: 32 - protein, 114 aa, synthetic construct
IEEIQKSIASIQESIASIQKSIYSMTGGSGGGSGGGSGGGSGMSIEEIQKQIAAIQKQIAAIQKQIYRMTTGGSGGGSGGGSGGGSGMSIEEIQKQISAISEQISAISKQISAM

SEQ ID NO: 33 - protein, 114 aa, synthetic construct
IEEIQKQIAAIQKQIAAIQKQIYAMTGGSGGGSGGGSGGGSGMSIEEIQKQIAAIQKQIAAIQKQIYRMTTGGSGGGSGGGSGGGSGMSIEEIQSQISSIQSQISSIQSQISSM

SEQ ID NO: 34 - protein, 93 aa, synthetic construct
IQQIQKSIASIQESIYSMTGGSGGGSGGGSGGGSGMDIQQIQKQIAAIQKQIYAMTTGGSGGGSGGGSGGGSGMDIQQIQKQISAISEQISAM

SEQ ID NO: 35 - protein, 66 aa, synthetic construct
MVKVKFKYKGEEKEVDTSKIKKVWRVGKMVSFTYDDNGKTGRGAVSEKDAPKELLDMLARAEREKK

SEQ ID NO: 36 - protein, 66 aa, synthetic construct
MVKVKFXXXGEEKEVDTSKIXXVXRXGKXVXFXYDDNGKXGXGXVXEKDAPKELLDMLARAEREKK

SEQ ID NO: 37 - protein, 66 aa, synthetic construct
MVKVKFSSSGEEKEVDTSKISSVSRSGKSVSFSYDDNGKSGSGSVSEKDAPKELLDMLARAEREKK

SEQ ID NO: 38 - protein, 89 aa, synthetic construct
VRAVPGNENSLEIEELARFAVDEHNKKENALLEFVRVVKAKEQVVAGTMYYLTLEAKDGGKKKLYEAKVMVKPWENFKELQEFKPVGDA

SEQ ID NO: 39 - protein, 100 aa, synthetic construct
VRAVPGNENSLEIEELARFAVDEHNKKENALLEFVRVVKAKEQXXXXXXXXXTMYYLTLEAKDGGKKKLYEAKVMVKXXXXXXXXXNFKELQEFKPVGDA

SEQ ID NO: 40 - protein, 100 aa, synthetic construct
VRAVPGNENSLEIEELARFAVDEHNKKENALLEFVRVVKAKEQSSSSSSSSSTMYYLTLEAKDGGKKKLYEAKVMVKSSSSSSSSSNFKELQEFKPVGDA

SEQ ID NO: 41 - protein, 90 aa, synthetic construct; identical to SEQ ID NO: 21

(19) United States
(12) Patent Application Publication
(10) Pub. No.: US 2025/0249112 A1
(43) Pub. Date: Aug. 7, 2025

(54) MACROPINOCYTOSIS SELECTIVE NON-BINDING PROTEIN-DRUG CONJUGATES

(71) Applicants: Craig RAMIREZ, Avon, CT (US); Andrew HAUSER, New York, NY (US); Tezcat Biosciences, Inc., Avon, CT (US)

(72) Inventors: Craig Ramirez, Avon, CT (US); Andrew Hauser, New York, NY (US)

(21) Appl. No.: 18/861,070

(22) PCT Filed: Apr. 28, 2023
(86) PCT No.: PCT/US2023/066375

§ 371 Date: Oct. 28, 2024

Related U.S. Application Data
(60) Provisional application No. 63/363,847, filed on Apr. 29, 2022.

(51) Int. Cl. A61K 47/68; A61P 35/00

(52) U.S. Cl. CPC A61K 47/68031; A61K 47/6807; A61K 47/6889; A61P 35/00

(57) ABSTRACT

Described herein are non-binding protein-drug conjugates having increased susceptibility to macropinocytosis by a population of cells in a macropinocytosis-positive disease state relative to a population of cells that are not in a macropinocytosis-positive disease state. The non-binding protein-drug conjugate can comprise a first portion comprising a non-binding protein scaffold that does not substantially bind to a cell surface, wherein the non-binding protein scaffold does not comprise a non-binding fibronectin type III (FN3) domain. A peptide linker can be coupled to and positioned between the first portion and a second portion, wherein the second portion can comprise a pharmaceutically active moiety or a diagnostic moiety.

### CROSS-REFERENCE TO RELATED APPLICATION

This application claims the priority of U.S. provisional application Ser. No. 63/363,847, filed Apr. 29, 2022, the disclosure of which is incorporated herein by reference in its entirety as if fully set forth herein.

### INCORPORATION BY REFERENCE OF SEQUENCE LISTING

A Sequence Listing is provided herewith as an xml file, “2329864.xml” created on Apr. 28, 2023 and having a size of 40,903 bytes. The content of the xml file is incorporated by reference herein in its entirety.

### BACKGROUND OF THE INVENTION

Macropinocytosis is an important mechanism of internalizing extracellular materials and dissolved molecules in eukaryotic cells. Macropinocytosis is a transient, non-selective, actin-dependent, endocytic process that leads to the internalization of fluid, membrane, nutrients, and other extracellular particles into large intracellular vacuoles. The regulatory factors involved in macropinocytosis include ADP ribosylation factor-6 (Arf6), actinin-4, p21-activated kinase 1 (PAK1), Cdc42, Rac, Rab GTPases, RhoA, and phosphoinositides (PIs).

Macropinocytosis plays a role in many pathologies, including cardiovascular disorders, cancer, allergic diseases, viral and bacterial infections. In certain cancer cells, including K-RAS-transformed cells, the cancer cells supply themselves with proteins, amino acids, lipids, and other nutrients by internalization through macropinocytosis to support rapid cell growth. Some cancer cells can also increase their own ATP levels by internalizing extracellular ATP through macropinocytosis. Macropinocytosis also has a role in the propagation of protein aggregation associated with neurodegenerative diseases such as Alzheimer's disease, Creutzfeldt-Jakob disease (CJD), Parkinson's disease, Huntington's disease, frontotemporal dementia (FTD), and amyotrophic lateral sclerosis (ALS).

There exists a need for developing therapeutics that are selectively engulfed through the enhanced macropinocytosis exhibited by pathological cells involved in disease processes.

### DESCRIPTION OF THE DRAWINGS

The drawings illustrate generally, by way of example, but not by way of limitation, various embodiments discussed herein.

FIGS. 1A-1B are graphs depicting the differential cytotoxicity of six non-binding protein-drug conjugates wherein the pharmaceutically active moiety is the cytotoxic chemotherapy drug monomethyl auristatin (MMAE) (referred to as non-binding protein-MMAE conjugates). FIG. 1A is a graph illustrating the cytotoxicity of the non-binding protein drug conjugates in HeLa KRasV12 cells. FIG. 1B is a graph illustrating the cytotoxicity of the non-binding protein drug conjugates in MiaPaCa-2 cells.

FIG. 2 is a graph depicting the IC₅₀ values of non-binding protein-MMAE conjugates wherein the pharmaceutically active moiety is MMAE. The IC₅₀ values were determined in HeLa and HeLa KRasV12 cells to determine if the Ras mutational status of the cells confers differential cytotoxicity by treatment with the non-binding protein-MMAE conjugates.

### SUMMARY

As illustrated herein, cells in a macropinocytosis-positive disease state can be targeted with a pharmaceutically active moiety or a diagnostic moiety using non-binding protein-drug conjugates. The non-binding protein-drug conjugate comprises a non-binding protein scaffold bound to a peptide linker and pharmaceutically active moiety or a diagnostic moiety also bound to the peptide linker. The non-binding protein scaffold can have increased susceptibility to macropinocytosis by a cell in a macropinocytosis-positive disease state as compared to a cell that is not in a macropinocytosis-positive disease state. As the non-binding protein scaffold is engulfed and internalized by such cells, the pharmaceutically active moiety or a diagnostic moiety attached thereto can be effective in destroying, modulating, or detecting the cells. Therefore, as demonstrated herein, the non-binding protein scaffold can act as a vehicle to selectively target cells in a macropinocytosis-positive disease state.

### DETAILED DESCRIPTION

Described herein are non-binding protein-drug conjugate that can comprise three portions: a non-binding protein scaffold, a peptide linker, and a pharmaceutically active moiety or a diagnostic moiety. The peptide linker may be bound to both the non-binding protein scaffold and the pharmaceutically active moiety or the diagnostic moiety to form the complete conjugate. The non-binding protein-drug conjugate can have increased susceptibility to macropinocytosis by a population of cells in a macropinocytosis-

positive disease state relative to a population of cells that are not in a macropinocytosis-positive disease state.

### Non-Binding Protein Scaffold

A first portion of the non-binding protein-drug conjugate can comprise a non-binding protein scaffold, which provides the non-binding protein-drug conjugate with its susceptibility to cellular uptake through macropinocytosis (MP). The non-binding protein scaffold may not include a non-binding fibronectin type III (FN3) domain. Examples of non-binding protein scaffolds are described in Examples 1-3 below.

The non-binding protein scaffold can be a synthetic or mutated native protein that does not bind any surface molecules on the surface of a cell. For example, the non-binding protein scaffold can be a protein that does not substantially bind material on the surface of a cell, such as proteins, lipids, or carbohydrates. A protein that does not substantially bind material on the cell surface is one that does not bind at all or may bind with transient nonspecific interactions. Such a non-binding protein does not interact with the material on the cell surface through forces such as electrostatic forces, hydrogen bonding, or the hydrophobic effect. The non-binding protein scaffold can be more than 5 kDa to increase susceptibility of the non-binding protein scaffold to being engulfed through macropinocytosis by the cell in the macropinocytosis-positive disease state.

The non-binding protein scaffold can be a protein scaffold with ligand-binding residues located in exposed flexible loops, wherein the ligand-binding residues are removed or mutated to become non-binding. Such protein scaffolds can include, but are not limited to, Affibody, Repebody, Affimer (Adhiron), Nanofitin (Affitin), Alphabody, Obodies, Kunitz domains, Fynomers, DARPin, Atrimer, Anticalin, or Affilin. Example sequences of these protein scaffolds are described below.

The non-binding protein scaffold can also be a protein scaffold with the binding residues located in protein secondary structures, such as α-helices, wherein the ligand-binding residues are removed or mutated to become non-binding. Such protein scaffolds can include, but are not limited to, affibodies, β-hairpin mimetics, and designed ankyrin repeat proteins (DARPins).

The non-binding protein scaffold with the binding residues in the protein secondary structure can be a modified antibody mimetic. Antibody mimetics are proteins engineered to bind to many target proteins or peptides with high affinity, imitating monoclonal antibodies. Antibody mimetics can be fused with peptide linker sequences, other proteins, or therapeutics through genetic modifications or post-translational spontaneous isopeptide bond formation. One or more binding regions of the protein sequence of the antibody mimetic can be mutated to render the binding region(s) nonfunctional while maintaining the secondary structure of the protein scaffold to provide susceptibility of the protein to cellular uptake specifically through MP.

The non-binding protein scaffold can comprise one or more amino acid substitutions in a protein binding sequence of an antibody-based protein scaffold. For example, the antibody-based protein scaffold can include an immunoglobulin, Fab, ScFv, Abdurin, Nanobody, or Humabody.

The non-binding protein scaffold can be thermostable and can be easily produced in microorganisms or completely synthesized chemically. In addition, non-binding protein scaffold can derive from human proteins and thus possess very low immunogenic potential.

### Peptide Linker

The non-binding protein-drug conjugate of the present disclosure further comprises the peptide linker that couples the first portion of the non-binding protein-drug conjugate to the second portion of the conjugate. The peptide can be a cleavable linker or a non-cleavable linker. Suitable peptide linkers can include peptides composed of repetitive modules of one or more of the amino acids, such as glycine and serine or alanine and proline. Exemplary linker peptides can include, e.g., (Gly-Gly) n, (Gly-Ser) n, (Gly3-Ser) n, (Ala-Pro) n wherein n is an integer from 1-25. The length of the peptide linker can be modified not to affect the function of the non-binding protein-drug conjugate. The standard 15 amino acid (Gly4-Ser) 3 peptide linker has been well-characterized and has been shown to adopt an unstructured, flexible conformation. In addition, this peptide linker does not interfere with assembly and activity of the domains it connects (Freund et al., “Characterization of the Linker Peptide of the Single-Chain Fv Fragment of an Antibody by NMR Spectroscopy,” FEBS 320:97 (1993), the disclosure of which is hereby incorporated by reference in its entirety).

In another example, valine-citrulline (Val-Cit) coupled with a self-immolative p-aminobenzyl (PAB) spacer can be used as a cleavable dipeptide linker, designated as “VC linker.” The VC linker is recognized and cleaved intracellularly by the dominant proteases found in a tumor cell lysosome, such as cathepsin B.

### Linking Method

Chemical coupling methods can be used to attach the first portion and second portion of the non-binding protein-drug conjugate using the peptide linker. For example, covalent conjugation of the first portion and the second portion can be accomplished via lysine side chains using an activated ester or isothiocyanate, or via cysteine side chains with a maleimide, haloacetyl derivative or activated disulfide. Site specific conjugation of the first portion and the second portion can also be accomplished by incorporating unnatural amino acids, self-labeling tags (e.g., SNAP or DHFR), or a tag that is recognized and modified specifically by another enzyme such as sortase A, lipoic acid ligase, and formylglycine-generating enzyme. In some embodiments, site specific conjugation of the first portion and the second portion is achieved by the introduction of cysteine residue either at the C-terminus of the non-binding protein scaffold or at a specific site as described by Goldberg et al., “Engineering a Targeted Delivery Platform Using Centyrins,” Protein Engineering, Design & Selection 29 (12): 563-572 (2016) and U.S. Patent Application Publication No. 20200325210 to Anderson et al., which are hereby incorporated by reference in their entirety.

### Pharmaceutically Active Moiety

In some embodiments, the second portion of the non-binding protein-drug conjugate as described herein is a pharmaceutically active moiety. Suitable pharmaceutically active moieties can include, but is not limited to, small molecules, nucleic acid molecules, antibodies, proteins or polypeptide fragments thereof, and a proteolysis targeting chimeras (PROTAC).

The pharmaceutically active moiety can be a cancer therapeutic. Suitable cancer therapeutics include, without limitation, an antimetabolite, an alkaloid, an alkylating agent, an anti-mitotic agent, an antitumor antibiotic, a DNA binding drug, a toxin, an antiproliferative drug, a DNA antagonist, a radionuclide, a thermoablative agent a proteolysis targeting chimera (PROTAC), a nucleic acid inhibitor, and an immune-modulatory agent.

The cancer therapeutic can be an alkaloid. Suitable alkaloids include, without limitation, duocarmycin, docetaxel, etoposide, irinotecan, paclitaxel, teniposide, topotecan, vinblastine, vincristine, vindesine, and analogs and derivatives thereof.

The cancer therapeutic can be an alkylating agent. Suitable alkylating agents include, without limitation, busulfan, improsulfan, piposulfan, benzodepa, carboquone, meturedepa, uredepa, altretamine, triethylenemelamine, triethylenephosphoramide, triethylenethiophosphorarnide, chlorambucil, chloranaphazine, cyclophosphamide, estramustine, ifosfamide, mechlorethamine, mechlorethamine oxide HCl, melphalan, novemebichin, perfosfamide phenesterine, prednimustine, trofosfamide, uracil mustard, carmustine, chlorozotocin, fotemustine, lomustine, nimustine, semustine ranimustine, dacarbazine, mannomustine, mitobronitol, mitolactol, pipobroman, temozolomide, and analogs and derivatives thereof.

The cancer therapeutic can be an antitumor antibiotic. Suitable antitumor antibiotics include, without limitation, aclacinomycin, actinomycin, anthramycin, azaserine, bleomycin, cactinomycin, calicheamicin, carubicin, carzinophilin, cromomycin, dactinomycin, daunorubicin, 6-diazo-5-oxo-1-norleucine, doxorubicin, epirabicin, idarubicin, menogaril, mitomycin, mycophenolic acid, nogalamycine, olivomycin, peplomycin, pirarubicin, plicamycin, porfiromycin, puromycine, pyrrolobenzodiazepine, streptonigrin, streptozocin, tubercidin, zinostatin, zorubicin, and analogs and derivatives thereof.

The cancer therapeutic can be an antimetabolite agent. Suitable antimetabolite agents include, without limitation, SN-38, denopterin, edatrexate, mercaptopurine (6-MP), methotrexate, piritrexim, pteropterin, pentostatin (2′-DCF), tomudex, trimetrexate, cladridine, fludarabine, thiamiprine, ancitabine, azacitidine, 6-azauridine, carmofur, cytarabine, doxifluridine, emitefur, floxuridine, fluorouracil, gemcitabine, tegafur, hydroxyurea, urethane, and analogs and derivatives thereof.

The cancer therapeutic can be an anti-proliferative drug. Suitable anti-proliferative drugs include, without limitation, aceglatone, amsacrine, bisantrene, camptothecin, defosfamide, demecolcine, diaziquone, diflomotecan, eflornithine, elliptinium acetate, etoglucid, etopside, fenretinide, gallium nitrate, hydroxyurea, lamellarin D, lonidamine, miltefosine, mitoguazone, mitoxantrone, mopidamol, nitracrine, pentostatin, phenamet, podophillinic acid 2-ethyl-hydrazide, procarbazine, razoxane, sobuzoxane, spirogermanium, teniposide, tenuazonic acid, triaziquone 2,2′,2″-trichlorotriethylamine, and analogs and derivatives thereof.

The cancer therapeutic can be an antimitotic agent. Suitable antimitotic agents include, without limitation, auristatin, a maytansinoid, a dolastatin, a tubulysin, a taxane, a epothilone, a vinca alkaloid, and analogs and derivatives thereof. In some embodiments, the antimitotic agent is an auristatin. In some embodiments, the auristatin is monomethyl auristatin E (MMAE).

The cancer therapeutic can be a PROTAC. Suitable PROTACs include, without limitation BET degraders, such as that disclosed by Pillow et al., “Antibody Conjugation of a Chimeric BET Degrader Enables In vivo Activity,” ChemMedChem 15 (1): 17-25 (2020), which is hereby incorporated by reference in its entirety. Suitable PROTACs can also include degraders of proteins involved in the Ras pathway.

In some embodiments, the pharmaceutically active moiety of the non-binding protein-drug conjugate as described herein can be an immunomodulatory agent. The immunomodulatory agent can be an agent that modifies the phenotype of one or more types of immune cells, e.g., type-1 macrophages, type-2 macrophages, dendritic cells, neutrophils, B cells, and T cells. For example, the immunomodulatory agent can be an agent that modifies the phenotype of an immune cell to result in immune cell activation. The immunomodulatory agent can also be an agent that modifies the phenotype of an immune cell to result in immune cell suppression.

The immunomodulatory agent can also be a macrophage type-1 stimulating agent. Suitable macrophage type-1 stimulating agents include, without limitation, paclitaxel, a colony stimulating factor-1 (CSF-1) receptor antagonist, an IL-10 receptor antagonist, a Toll-like receptor (TLR)-2 agonist, a TLR-3 agonist, a TLR-4 agonist, a TLR-7 agonist, a TLR-8 agonist, and a TLR-9 agonist.

The macrophage type-1 stimulating agent can be a CSF-1 receptor antagonist. Suitable CSF-1 receptor antagonists include, without limitation ABT-869 (Guo et al., “Inhibition of Phosphorylation of the Colony-Stimulating Factor-1 Receptor (c-Fms) Tyrosine Kinase in Transfected Cells by ABT-869 and Other Tyrosine Kinase Inhibitors,” Mol. Cancer. Ther. 5 (4): 1007-1012 (2006), which is hereby incorporated by reference in its entirety), imatinib (Guo et al., “Inhibition of Phosphorylation of the Colony-Stimulating Factor-1 Receptor (c-Fms) Tyrosine Kinase in Transfected Cells by ABT-869 and Other Tyrosine Kinase Inhibitors,” Mol. Cancer. Ther. 5 (4): 1007-1012 (2006), which is hereby incorporated by reference in its entirety), PLX3397 (Mok et al., “Inhibition of CSF1 Receptor Improves the Anti-tumor Efficacy of Adoptive Cell Transfer Immunotherapy,” Cancer Res. 74 (1): 153-161 (2014), which is hereby incorporated by reference in its entirety), PLX5622 (Dagher et al., “Colony-stimulating Factor 1 Receptor Inhibition Prevents Microglial Plaque Association and Improves Cognition in 3xTg-AD Mice,” J. Neuroinflamm. 12:139 (2015), which is hereby incorporated by reference in its entirety), DCC-3014 (Deciphera Pharmaceuticals), BLZ945 (Krauser et al., “Phenotypic and Metabolic Investigation of a CSF-1R Kinase Receptor Inhibitor (BLZ945) and its Pharmacologically Active Metabolite,” Xenobiotica 45 (2): 107-123 (2015), which is hereby incorporated by reference in its entirety), and GW2580 (Olmos-Alonso et al., “Pharmacological Targeting of CSFIR Inhibits Microglial Proliferation and Prevents the Progression of Alzheimer's-like Pathology,” Brain 139:891-907 (2016), which is hereby incorporated by reference in its entirety.

The macrophage type-1 stimulating agent can be an IL-10 receptor antagonist. Suitable IL-10 receptor antagonists include, without limitation, peptide antagonists as described in Naiyer et al., “Identification and Characterization of a Human IL-10 Receptor Antagonist,” Hum. Immunol. 74 (1): 28-31 (2013), which is hereby incorporated by reference in its entirety, and IL-10 receptor antagonistic antibodies as described in U.S. Pat. No. 7,553,932 to Von Herrath et al., which is hereby incorporated by reference in its entirety.

The macrophage type-1 stimulating agent can be a TLR agonist, i.e., a TLR2, TLR3, TLR4, TLR7, TLR8, or TLR9 agonist. Suitable TLR-2 agonists for use in the methods described herein include, without limitation, Pam3CSK4, a synthetic triacylated lipoprotein, and lipoteichoic acid (LTA) (Brandt et al., “TLR2 Ligands Induce NF-κB Activation from Endosomal Compartments of Human Monocytes” PLOS One 8 (12): e80743, which is hereby incorporated by reference in its entirety). A suitable TLR-3 agonist includes, without limitation, polyinosinic: polycytidylic acid (poly I:C) (Smole et al., “Delivery System for the Enhanced Efficiency of Immunostimulatory Nucleic Acids,” Innate Immun. 19 (1): 53-65 (2013), which is hereby incorporated by reference in its entirety). Suitable TLR-4 agonists include, without limitation, MPL (Engel et al., “The Pharmacokinetics of Toll-like Receptor Agonists and the Impact on the Immune System,” Expert Rev. Clin. Pharmacol. 4 (2): 275-289 (2011), which is hereby incorporated by reference in its entirety), Glucopyranosyl Lipid-A (Matzner et al., “Perioperative treatment with the new synthetic TLR-4 agonist GLA-SE reduces cancer metastasis without adverse effects,” Int. J. Cancer 138 (7): 1754-64 (2016), which is hereby incorporated by reference in its entirety), and Immunomax® (Ghochikyan et al., “Targeting TLR-4 with a novel pharmaceutical grade plant derived agonist, Immunomax®, as a therapeutic strategy for metastatic breast cancer,” J. Trans. Med. 12:322 (2014), which is hereby incorporated by reference in its entirety).

Suitable TLR-7 agonists can include, without limitation, uridine/guanidine-rich single-stranded RNA (Engel et al., “The Pharmacokinetics of Toll-like Receptor Agonists and the Impact on the Immune System,” Expert Rev. Clin. Pharmacol. 4 (2): 275-289 (2011), which is hereby incorporated by reference in its entirety), 852A (Dudek et al., “First in Human Phase I Trial of 852A, a Novel Systemic Toll-like Receptor 7 Agonist, to Activate Innate Immune Responses in Patients With Advanced Cancer,” Clin. Cancer Res. 13 (23): 7119-7125 (2007), which is hereby incorporated by reference in its entirety), resiquimod (Chang et al., “Topical resiquimod Promotes Priming of CTL to Parenteral Antigens,” Vaccine 27 (42): 5791-5799 (2009), which is hereby incorporated by reference in its entirety), imidazoquinolines (Itoh et al., “The Clathrin-mediated Endocytic Pathway Participates in dsRNA-induced IFN-beta Production,” J. Immunol. 181:5522-9 (2008), which is hereby incorporated by reference in its entirety), ANA975 (Fletcher et al., “Masked oral Prodrugs of Toll-like Receptor 7 Agonists: a New Approach for the Treatment of Infectious Disease,” Curr. Opin. Investig. Drugs 7 (8): 702-708 (2006), which is hereby incorporated by reference in its entirety), and imiquimod (Engel et al., “The Pharmacokinetics of Toll-like Receptor Agonists and the Impact on the Immune System,” Expert Rev. Clin. Pharmacol. 4 (2): 275-289 (2011), which is hereby incorporated by reference in its entirety).

Suitable TLR-8 agonists can include, without limitation, resiquimod (Chang et al., “Topical resiquimod Promotes Priming of CTL to Parenteral Antigens,” Vaccine 27 (42): 5791-5799 (2009), which is hereby incorporated by reference in its entirety), and imidazoquinolines (Itoh et al., “The Clathrin-mediated Endocytic Pathway Participates in dsRNA-induced IFN-beta Production,” J. Immunol. 181:5522-9 (2008), which is hereby incorporated by reference in its entirety).

Suitable TLR-9 agonists can include, without limitation, CpG-ODN (Yao et al., “Late Endosome/Lysosome-localized Rab7b Suppresses TLR-9-initiated Proinflammatory Cytokine and Type I IFN Production in Macrophages,” J. Immunol. 183:1751-8 (2009), which is hereby incorporated by reference in its entirety). Specific CpG-ODNs suitable for use are described in Engel et al., “The Pharmacokinetics of Toll-like Receptor Agonists and the Impact on the Immune System,” Expert Rev. Clin. Pharmacol. 4 (2): 275-289 (2011), which is hereby incorporated by reference in its entirety.

Other agents known in the art to reprogram type-2 macrophages to type-1 macrophages (i.e., macrophage type-1 stimulating agent) include, without limitation, manganese dioxide nanoparticles (see e.g., Song et al., “Bioconjugated Manganese Dioxide Nanoparticles Enhance Chemotherapy Response by Priming Tumor-Associated Macrophages toward M1-like Phenotype and Attenuating Tumor Hypoxia” ACS Nano. 10:633-647 (2016), which is hereby incorporated by reference in its entirety), ferumoxytal nanoparticles (Zanganeh, et al. “Iron oxide nanoparticles inhibit tumour growth by inducing pro-inflammatory macrophage polarization in tumour tissues,” Nat. Nanotechnol. 11:986-994 (2016), which is hereby incorporated by reference in its entirety), mannosylated nanoparticles encapsulating siRNA against IκBα (Ortega et al. “Manipulating the NF-kappaB pathway in macrophages using mannosylated, siRNA-delivering nanoparticles can induce immunostimulatory and tumor cytotoxic functions,” Int. J. Nanomed. 2163-2177 (2016), which is hereby incorporated by reference in its entirety). In accordance with the present disclosure, these agents can be coupled to a non-binding protein scaffold via a peptide linker as described herein to form a non-binding protein-drug conjugate.

The immunomodulatory agent can be a macrophage type-2 stimulating agent. Suitable macrophage type-2 stimulating agents include, without limitation, IL-33, IL-4 receptor agonists, glucocorticoids, IL-10 receptor agonists, and IL-1 receptor agonists.

Suitable IL-4 receptor agonists can include, without limitation, mutant IL-4 proteins. Exemplary mutant IL-4 proteins include, but are not limited to those described in U.S. Pat. No. 5,723,118 to Sebald, which is hereby incorporated by reference in its entirety.

Glucocorticoids are a class of corticosteroids, which are well known in the art and suitable for inducing a macrophage type-2 phenotype. Exemplary glucocorticoids for incorporation into the non-binding protein-drug conjugate of the present disclosure can include, without limitation, cortisol, cortisone, prednisone, prednisolone, methylprednisolone, dexamethasone, betamethasone, triamcinolone, beclomethasone, fludrocortisone, deoxycorticosterone, and aldosterone.

IL-10 receptor agonists are also capable of inducing a macrophage type-2 phenotype in accordance with the conjugates and methods described herein. Suitable IL-10 receptor agonists can include, without limitation, mutant IL-10 proteins as described in U.S. Pat. No. 7,749,490 to Sommer et al., which is hereby incorporated by reference in its entirety.

IL-1 receptor agonists are also capable of inducing a macrophage type-2 phenotype and, therefore, can be incorporated as the drug component of the non-binding protein-conjugates described herein. Suitable IL-1 receptor agonists can include, without limitation, IL-1α, IL-1β, IL-18, IL-33, IL-36α, IL-36β, and IL-36γ (Palomo et al., “The Interleukin (IL)-1 Cytokine Family-Balance Between Agonists and Antagonists in Inflammatory Diseases,” Cytokine 76 (1): 25-37 (2015), which is hereby incorporated by reference in its entirety).

The immunomodulatory agent can be a macrophage type-2 depleting agent. Suitable macrophage depleting agents include, without limitation, clodronate, zoledronic acid, alendronate, and trabectedin.

The immunomodulatory agent can be a T cell stimulating agent. Suitable T cell stimulating agents include, without limitation stimulator of interferon genes (STING) agonists. STING agonists include, without limitation, cyclic dinucleotides (CDNs), such as cyclic dimeric guanosine monophosphate (c-di-GMP), cyclic dimeric adenosine monophosphate (c-di-AMP), cyclic GMP-AMP (cGAMP), and dithio-(RP,RP)-[cyclic[A(2′,5′)pA(3′,5′)p (ADU-S100, Aduro Biotech) and small molecules, such as 5,6-dimethylxanthenone-4-acetic acid (DMXAA) and linked amidobenzimidazole. Other STING agonists under development that are also suitable immunomodulatory agents in accordance with the present disclosure include BMS-986301, E7766, GSK3745417, MK-1454, MK-2118, and SB11285.

The immunomodulatory agent can be a dendritic cell stimulating agent. Suitable dendritic cell stimulating agents for inclusion in the non-binding protein-drug conjugate as described herein can include, without limitation, CpG oligonucleotide, imiquimod, topoisomerase I inhibitors (e.g., camptothecin and derivatives thereof), microtubule depolymerizing drugs (e.g., colchicine, podophyllotoxin, and derivatives thereof).

In some embodiments, the immunomodulatory agent can be a neutrophil stimulating agent. Suitable neutrophil stimulating agents include, without limitation, recombinant granulocyte colony stimulating factor protein (filgrastim) or a pegylated recombinant granulocyte colony stimulating factor protein.

In some embodiments, the pharmaceutically active moiety of the non-binding protein-drug conjugate of the present disclosure can be an oligonucleotide. Suitable oligonucleotides include, without limitation, an siRNA, an aptamer, an miRNA, an immunostimulatory oligonucleotide, a splice-switching oligonucleotide, and guide RNA.

The pharmaceutically active moiety can be a wound healing agent. Suitable wound healing agents in accordance with this aspect of the disclosure include, without limitation, an agent that stimulates a proinflammatory phenotype of an immune cell. In some embodiments, the pharmaceutically active moiety for the treatment of wound healing can be a macrophage type-1 stimulating agent as described supra.

### Diagnostic Moiety

The second portion of the non-binding protein-drug conjugate of the present disclosure can be a diagnostic moiety. Suitable diagnostic moieties are those that facilitate the detection, quantitation, separation, and/or purification of the non-binding protein-drug conjugate. Suitable diagnostic moieties can include, without limitation, purification tags (e.g., poly-histidine (His6-), glutathione-S-transferase (GST-), maltose-binding protein (MBP-)), fluorescent dyes or tags (e.g., chelates (europium chelates), fluorescein and its derivatives, rhodamine and its derivatives, dansyl, Lissamine, phycoerythrin and Texas Red, an enzymatic tag, a radioisotope or radioactive label (e.g., 4C, 11C, 14N, 35S, 3H, 32P, 99mTc, 111 In, 62/64Cu, 125I, 18F, 67/68Ga, 90Y, 177Lu and 186/188Re), a radionucleotide with chelator (e.g., MAG3, DTPA, and DOTA, see also, Liu S., “Bifunctional Coupling Agents for Radiolabeling of Biomolecules and Target Specific Delivery of Metallic Radionuclides,” Adv. Drug Deli. Ref. 60 (12): 1347-1370 (2008), which is hereby incorporated by reference in its entirety), a microbubble (Abou-Elkacem et al., “Ultrasound molecular imaging of the breast cancer neovasculature using engineered fibronectin scaffold ligands: A novel class of targeted contrast ultrasound agent,” Theranostics 6:1740-1752 (2016), which is hereby incorporated by reference in its entirety), a contrast agent suitable for imaging, or a photosensitize.

The diagnostic moiety can be a radiolabel, radionuclide or radioisotope bound to a chelating agent. Particularly useful diagnostic radiolabels, radionuclides, or radioisotopes that can be bound to a chelating agent include, without limitation, 110In, mIn, 177Lu, 18F, 52Fe, 62Cu, 64Cu, 67Cu, 67Ga, 68Ga, 86Y, 9 V, 89Zr, 94Tc, 94Tc, 99mTc, 120I, 123I, 124I, 125I, 131I, 154Gd, 158Gd, 32P, nC, 13N, 150, 186Re, 188Re, 51Mn, 52mMn, 55Co, 72As, 75Br, 76Br, 82mRb, 83Sr, or other gamma-, beta-, or positron-emitters. The diagnostic radiolabels include a decay energy in the range of 25 to 10,000 keV, more preferably in the range of 25 to 4,000 keV, and even more preferably in the range of 20 to 1,000 keV, and still more preferably in the range of 70 to 700 keV. Total decay energies of useful positron-emitting radionuclides are preferably <2,000 keV, more preferably under 1,000 keV, and most preferably <700 keV.

Chelators such as NOTA (1, 4, 7-triaza-cyclononane-N,N′,N″-triacetic acid), DOTA (1, 4, 7, 10-tetraazacyclododecane-1, 4, 7, 10-tetraacetic acid), DTP A (1, 1, 4, 7, 7-Diethylenetriaminepentaacetic acid), TETA (p-bromoacetamido-benzyl-tetraethylaminetetraacetic acid), and Df (desferrioxamine B) are of use with a variety of radiolabels, radionuclides, radioisotopes, metals and radiometals. DOTA-type chelators, where the ligand includes hard base chelating functions such as carboxylate or amine groups, are most effective for chelating hard acid cations. Such metal-chelate complexes can be made very stable by tailoring the ring size to the metal of interest. Also, more than one type of chelator may be conjugated to the targetable construct to bind multiple metal ions, e.g., diagnostic radionuclides and/or therapeutic radionuclides.

Chelators can be covalently bound to the non-binding protein scaffold using standard methods of bioconjugation. Amine containing residues (e.g., lysine) in the non-binding protein scaffold can undergo amide bond formation with a chelator containing an activated ester (e.g., an N-hydroxysuccinimidyl ester). Sulfur containing residues (e.g., cysteine) undergo conjugation with chelators containing an activated ester or maleimide moiety. Alternatively, bioconjugates can be formed when activated carboxylate residues of the non-binding protein scaffold undergo amide or thoiester formation with amine or thiol groups, respectively, on the chelator. Bifunctional linkers, such as, for example, PEG-maleimide (PEG-Mal), succinimidyl-4-(N-maleimidomethyl) cyclohexane-1-carboxylate (SMCC) or N-succinimidyl 3-(2-pyridylthio) propionate (SPDP) can be alternatively used.

### Half-Life Extending Moiety

In some embodiments, the non-binding protein-drug conjugate of the present disclosure can further comprise a third portion. The third portion of the non-binding protein-drug conjugate of the present disclosure can comprise a half-life extending moiety. Exemplary half-life extending moieties can include, without limitation, albumin, albumin variants (see e.g., U.S. Pat. No. 8,822,417 to Andersen et al., U.S. Pat. No. 8,314,156 to Desai et al., and U.S. Pat. No. 8,748,380 to Plumridge et al., which are hereby incorporated by reference in their entirety), albumin-binding proteins and/or domains, transferrin and fragments and analogues thereof (see e.g., U.S. Pat. No. 7,176,278 to Prior et al., which are hereby incorporated by reference in their entirety), Fc regions and variant Fc regions (see e.g., U.S. Pat. No. 8,546,543 to Lazar et al., U.S. Patent Publication No. 20150125444 to Tsui, and U.S. Pat. No. 8,722,615 to Seehra et al., which are hereby incorporated by reference in their entirety).

Other half-life extending moieties of the non-binding protein-drug conjugate can include, without limitation, polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. A pegyl moiety can, for example, be added to the first portion, i.e., non-binding protein scaffold, by adding a cysteine residue to the C-terminus of the molecule and attaching a pegyl group to the cysteine using methods well known in the art.

### Pharmaceutical Preparations

For therapeutic or diagnostic use, the non-binding protein-drug conjugates as described herein are prepared as pharmaceutical or diagnostic compositions containing an effective amount of the protein-drug conjugate as an active ingredient in a pharmaceutically acceptable carrier. The term “carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of non-binding protein-drug conjugate as described herein in such pharmaceutical formulation can vary widely, i.e., from less than about 0.5%, usually at or at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. REMINGTON: THE SCIENCE AND PRACTICE OF PHARMACY, 21st Edition, Troy, D. B. ed., Lipincott Williams and Wilkins, 2006, Part 5, Pharmaceutical Manufacturing pp 691-1092, see especially pp. 958-989, which is hereby incorporated by reference in its entirety.

The non-binding protein-drug conjugates described herein can be used in non-isolated or isolated form. Furthermore, the non-binding protein-drug conjugates described herein can be used alone or in a mixture comprising at least one other non-binding protein-drug conjugate as described herein. In other words, the non-binding protein-drug conjugates can be used in combination, e.g., as a pharmaceutical composition comprising two or more non-binding protein-drug conjugates. For example, non-binding protein-drug conjugates having different, but complementary activities can be combined in a single therapy to achieve a desired therapeutic effect, but alternatively, non-binding protein-drug conjugates having identical activities can also be combined in a single therapy to achieve a desired therapeutic or diagnostic effect. Optionally, the mixture further comprises at least one other therapeutic agent.

### Methods

Methods for treatment of a subject with a disease characterized by cells in a macropinocytosis-positive disease state may comprise administering the non-binding protein-drug conjugates to the subject. The second portion of the non-binding protein-drug conjugate comprising the pharmaceutically active moiety can target the disease through cellular uptake specifically through MP by cells in the macropinocytosis-positive disease state.

In accordance with the methods described herein a “subject” refers to any animal. The subject can be mammal including, without limitation, humans, non-human primates, dogs, cats, rodents (e.g., mouse, rat, guinea pig), horses, cattle and cows, sheep, and pigs.

### Cancer Treatment

The non-binding protein-drug conjugate can target cancer cells characterized by enhanced macropinocytosis relative to their corresponding non-cancerous cells. The cancerous cells can have an oncogenic mutation in RAS genes, i.e., an oncogenic mutation in H-ras, N-ras, or K-ras. The subject can have a cancer with the oncogenic mutation in RAS genes that can be, without limitation, pancreatic cancer, lung cancer, breast cancer, colon cancer, glioma, solid tumor, melanoma, glioblastoma multiforme, leukemia, renal cell carcinoma, hepatocellular carcinoma, prostate cancer, and myeloma.

The subject can have a type of cancer that is or has become resistant to primary cancer therapeutic treatment, e.g., resistant to chemotherapy treatment, prior to administering the non-binding protein-drug conjugate. The non-binding protein-drug conjugate can be administered in an amount effective to directly target and kill cancerous cells. The non-binding protein-drug conjugate can comprise any suitable cancer therapeutic, such as an antimetabolite, an alkaloid, an alkylating agent, an anti-mitotic agent, an antitumor antibiotic, a DNA binding drug, a microtubule targeting drug, a toxin, an antiproliferative drug, a DNA antagonist, radionuclide, a thermoablative agent or a PROTAC are described supra.

The subject can have a type of cancer that is or has become immune tolerant. Administering the non-binding protein-drug conjugate wherein the second portion comprises an immunomodulatory agent can be carried out in an amount effective to enhance the antitumor immune response. The non-binding protein-drug conjugate can comprise any suitable immunomodulatory agent, such as a macrophage type-1 stimulating agent, a macrophage type-2 depleting agent, a T cell stimulating agent, a dendritic cell stimulating agent, and/or a neutrophil stimulating agent are described supra.

Methods of treating a subject having cancer can further involve administering an additional cancer therapeutic in conjunction with the non-binding protein-drug conjugate. Suitable cancer therapeutics that can be administered in combination with the non-binding protein-drug conjugates described herein as a combination therapy include, for example and without limitation, chemotherapeutic agents. Suitable chemotherapeutics include, without limitation, alkylating agents (e.g., chlorambucil, cyclophophamide, CCNU, melphalan, procarbazine, thiotepa, BCNU, and busulfan), antimetabolites (e.g., methotrexate, 6-mercaptopurine, and 5-fluorouracil), anthracyclines (daunorubicin, doxorubicin, idarubicin, epirubicin, and mitoxantrone), antitumor antibiotics (e.g., bleomycin, monoclonal antibodies (e.g., Alemtuzumab, Bevacizumab, Cetuximab, Gemtuzumab, Ibritumomab, Panitumumab, Rituximab, Tositumomab, and Trastuxmab), platiniums (e.g., cisplatin and oxaliplatin) or plant alkaloids (e.g., topoisomerase inhibitors, vinca alkaloids, taxanes (e.g. paclitaxel), and epipodophyllotoxins). In some embodiments, the cancer chemotherapeutic is selected from cyclophosphamide, gemcitabine, vorinostat, temozolomide, bortezomib, carmustine, and paclitaxel.

In accordance with the methods described herein, administration of the non-binding protein-drug conjugates, alone or in combination with one or more additional cancer therapeutics, can be carried out by systemic or local administration. Suitable modes of systemic administration of the non-binding protein-drug conjugates with or without the cancer therapeutics can include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intra-arterially, intralesionally, or by application to mucous membranes. Suitable modes of local administration of the therapeutic agents and/or combinations disclosed herein include, without limitation, catheterization, implantation, direct injection, dermal/transdermal application, or portal vein administration to relevant tissues, or by any other local administration technique, method or procedure generally known in the art. The mode of affecting delivery can vary depending on the type of cancer therapeutic being delivered and the type of cancer to be treated.

A therapeutically effective amount of the non-binding protein-drug conjugate, alone or in combination with an additional cancer therapeutic, in the methods disclosed herein is an amount that, when administered over a particular time interval, results in achievement of one or more therapeutic benchmarks (e.g., slowing or halting of tumor growth, tumor regression, cessation of symptoms, etc.). The non-binding protein-drug conjugate can be administered to a subject one time or multiple times. In those embodiments where the therapeutic composition is administered multiple times, it may be administered at a set interval, e.g., daily, every other day, weekly, or monthly. Alternatively, it can be administered at an irregular interval, for example on an as-needed basis based on symptoms, patient health, and the like. For example, a therapeutically effective amount may be administered once a day (q.d.) for one day, at least 2 days, at least 3 days, at least 4 days, at least 5 days, at least 6 days, at least 7 days, at least 10 days, or at least 15 days. Optionally, the status of the cancer or the regression of the cancer can be monitored during or after the treatment, for example, by a multiparametric ultrasound (mpUS), multiparametric magnetic resonance imaging (mpMRI), and nuclear imaging (positron emission tomography [PET]) of the subject. The dosage of the non-binding protein-drug conjugate or combination therapy administered to the subject can be increased or decreased depending on the status of the cancer or the regression of the cancer detected.

The skilled artisan can readily determine this amount, on either an individual subject basis (e.g., the amount of a non-binding protein-drug conjugate necessary to achieve a particular therapeutic benchmark in the subject being treated) or a population basis (e.g., the amount of non-binding protein-drug conjugate necessary to achieve a particular therapeutic benchmark in the average subject from a given population). Ideally, the therapeutically effective amount does not exceed the maximum tolerated dosage at which 50% or more of treated subjects experience side effects that prevent further drug administrations.

A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like.

Another aspect of the present disclosure is directed to a method of modulating a subject's immune response. This method involves administering to the subject having a condition that would benefit from immune system modulation, a non-binding protein-drug conjugate as described herein in an amount effective to modulate the subject's immune response. In accordance with this aspect of the disclosure, suitable non-binding protein-drug conjugates include those conjugates comprising an immunomodulatory agent as the second portion of the conjugate. Suitable immunomodulatory agents can include, without limitation, type-1 macrophage stimulating agents, type-2 macrophage stimulating agents, T cell stimulating agents, dendritic cell stimulating agents, and neutrophil stimulating agents, as described supra.

Modulating or modifying a subject's immune response in accordance with this aspect of the disclosure can be for the purpose of treating, preventing, or slowing the progression of a disease or condition that is caused or exacerbated, at least in part, by the immune response and/or cells of the immune system, e.g., type-1 macrophages, type-2 macrophages, T cells, B cells, dendritic cells, neutrophils. For example, inflammatory diseases and conditions, including but not limited to macular degeneration, atherosclerosis, osteoporosis, immune inflammation, non-immune inflammation, renal inflammation, tuberculosis, multiple sclerosis, arthritis, chronic obstructive pulmonary disease (COPD), and Alzheimer's disease, involve the undesired actions of type-1 macrophages. Employing the methods of the present invention to induce a macrophage type-2 phenotypic change in the type-1 pro-inflammatory macrophages that are involved in or contributing to these disease processes can alleviate one or more symptoms or causes of the disease. Accordingly, in one embodiment, the administering can be carried out in vivo or ex vivo to a population of type-1 macrophages in or from a subject having an inflammatory or autoimmune condition, including, but not limited to any of those enumerated above. Administering a type-2 macrophage stimulating agent to a population of type-1 macrophages in this context will induce a type-2 phenotypic change, thereby reducing the undesired actions of the type-1 macrophages associated with the disease.

Modulating or modifying immune cell phenotype can also be therapeutically beneficial in context of treating various forms of cancer. Recent studies indicate that tumor-associated macrophages (TAMs) exhibit a macrophage type-2-like phenotype. These type-2 macrophages are important tumor-infiltrating cells and play pivotal roles in tumor growth and metastasis. In most solid tumors, the existence of TAMs is advantageous for tumor growth and metastasis. These TAMs produce interleukin IL-10 and transforming growth factor (TGF) β to suppress general antitumor immune responses. Meanwhile, TAMs promote tumor neo-angiogenesis by the secretion of pro-angiogenic factors and define the invasive microenvironment to facilitate tumor metastasis and dissemination. Therefore, administering the non-binding protein-drug conjugate comprising a macrophage type-1 stimulating agent to induce a type-1 phenotypic change in the TAMs to enhance anti-tumor immunity can significantly alter the progression of the cancer. Cancers that typically have a type-2 macrophage-related component include, without limitation, pancreatic cancer, breast cancer, and non-small cell lung cancer. Alternatively, or in conjunction with administering a non-binding protein-drug conjugate comprising a macrophage type-1 stimulating agent, a non-binding protein-drug conjugate comprising a T cell stimulating agent, a dendritic cell simulating agent, or a neutrophil stimulating agent can be administered to activate or enhance the antitumor immune response.

In some embodiments, the subject in need of immune system modulation can be suffering from an interferonopathy. As referred to herein, an interferonopathy is a condition involving the enhanced expression of type I interferons, e.g., IFN-α, IFN-β, and IFN-Q. Interferonopathies that can be treated in accordance with the present disclosure include, without limitation, Aicardi-Goutieres syndrome, Cree encephalitis, systemic lupus erythematosus, s, rheumatoid arthritis, Sjögrens syndrome, dermatomyositis, multiple sclerosis, spondyloenchondrodysplasia with immune dysregulation, stimulator of interferon genes (STING)-associated vasculopathy with onset in infancy (SAVI), Japanese autoinflammatory syndrome with lipodystrophy (JASL), ubiquitin-specific peptidase 18 deficiency, chronic atypical neutrophilic dermatitis with lipodystrophy, DNA II deficiency, Singleton-Merten syndrome, and chronic atypical neutrophilic dermatosis with lipodystrophy and elevated temperature (CANDLE). Non-binding protein-drug conjugates according to the present disclosure suitable for treating an interferonopathy can comprise the non-binding protein scaffold coupled to a type I interferon antagonist. In some embodiments, the type I interferon antagonist is a type I interferon receptor antagonist, such as a janus kinase (JAK) inhibitor. Suitable JAK inhibitors include, JAK1/JAK2 inhibitors, such as and without limitation baricitinib (CAS No. 1187594 Sep. 7), tofacitinib (CAS No. 477600-75-2), ruxolitinib (941678-49-5), AG490 (Tyrphostin family) (CAS No. 133550-30-8), Lestaurtinib (CEP-701; CAS No. 111358-88-4), WP-1034 (Tyrphostin family; CAS No. 857064-42-7), BMS-911543 (CAS No. 1271022-90-2), Fedratinib (TG101348; CAS No. 936091-26-8), Parcritinib (SB1518; CAS No. 937272-79-2), and Momelotinib (CYT387; CAS No. 1056634-68-4).

In some embodiments, the subject in need of immune system modulation can have a wound or be undergoing conventional medical procedures for wound healing. In accordance with this embodiment, the subject having a wound or in need of wound healing can be administered the non-binding protein-drug conjugate comprising a macrophage type-1 stimulating agent. Suitable type-1 stimulating agents, e.g., paclitaxel, a colony stimulating factor-1 (CSF-1) receptor antagonist, an IL-10 receptor antagonist, a Toll-like receptor (TLR)-2 agonist, a TLR-3 agonist, a TLR-4 agonist, a TLR-7 agonist, a TLR-8 agonist, and a TLR-9 agonist, are described supra.

In some embodiments, the subject in need of immune system modulation can have an inflammatory condition. The non-binding protein-drug conjugate comprising a macrophage depleting agent can be administered to the subject. Suitable inflammatory conditions that can be treated with a macrophage depleting agent, e.g., clodronate, zoledronic acid, and trabectedin, include, without limitation, rheumatoid arthritis, obesity and obesity related complications, endometriosis, inflammatory conditions of the lung (e.g., chronic obstructive pulmonary disease and pulmonary tuberculosis).

Another aspect of the present disclosure is directed to a method of imaging a tumor in a subject. This method involves selecting a subject having a tumor and administering to the subject the non-binding protein-drug conjugate comprising a diagnostic moiety. Suitable diagnostic moieties are described supra, e.g., fluorescent dyes, radioisotopes, radionuclides, radioisotopes, microbubbles, a contrast agent suitable for imaging, and a photosensitizer.

In accordance with this aspect of the disclosure, the tumor to be imaged can be include cancerous cells having enhanced macropinocytosis relative to their corresponding non-cancerous cells. In some embodiments, the tumor to be imaged can be characterized by cancerous cells having an oncogenic mutation in H-ras, N-ras, or K-ras. In some embodiments, the tumor to be imaged can be a pancreatic tumor, lung tumor, breast tumor, colon tumor, glioma, solid tumor, melanoma, glioblastoma multiforme, leukemia, renal cell carcinoma, hepatocellular carcinoma, prostate tumor, and myeloma.

Detecting the presence of a tumor in a subject using the diagnostic non-binding protein-drug conjugate can be achieved using in vivo imaging techniques. In vivo imaging involves administering to the subject the non-binding protein-diagnostic moiety conjugate described herein, and detecting the tumor cell macropinocytotic-mediated uptake of the conjugate in vivo.

In accordance with this aspect of the disclosure, diagnostic non-binding protein-drug conjugate can be administered by intravenous injection into the body of the subject, or directly into the tumor. The dosage of the diagnostic non-binding protein-drug conjugate can be within the same ranges as for treatment methods. The diagnostic moiety can be an imaging agent that facilitates in vivo imaging. Suitable imaging agents are described supra and include, without limitation, single photon emission computed tomography (SPECT) agents, positron emission tomography (PET) agents, magnetic resonance imaging (MRI) agents, nuclear magnetic resonance imaging (NMR) agents, x-ray agents, optical agents (e.g., fluorophores, bioluminescent probes, near infrared dyes, quantum dots), ultrasound agents and neutron capture therapy agents, computer assisted tomography agents, two photon fluorescence microscopy imaging agents, and multi-photon microscopy imaging agents. Exemplary detectable markers include radioisotypes (e.g., ¹⁸F, ¹¹C, ¹³N, ⁶⁴Cu, ¹²⁴I, ⁷⁶Br, ⁸²Rb, ⁶⁸Ga ⁹⁹mTc, ¹¹¹In, ²⁰¹Tl or ¹⁵O, which are suitable for PET and/or SPECT use) and ultra-small superparamagnetic particles of iron oxide (USPIO) which are suitable for MRI.

Imaging of a tumor can be performed by detecting the number, size, and/or intensity of detected non-binding protein-drug conjugate s in the subject. In some embodiments, the level of non-binding protein-drug conjugate can be compared to a corresponding baseline value. An appropriate baseline value can be the average level of non-binding protein diagnostic conjugate found within cells in a population of non-diseased individuals. Alternatively, an appropriate baseline value may be the level of non-binding protein-drug conjugate found within cells of the same subject determined at an earlier time.

The diagnostic imaging methods described herein can also be used to monitor a subject's response to therapy. In this embodiment, detection of the non-binding protein-drug conjugate in the subject can be determined prior to the commencement of treatment. The level of non-binding protein diagnostic conjugate in the subject at this time point can be used as a baseline value. At various times during the course of treatment, administration and detection of the non-binding protein-drug conjugate can be repeated, and the measured values thereafter compared with the baseline values. A decrease in values relative to baseline can signal a positive response to treatment.

### Dosages, Formulations and Routes of Administration

Pharmaceutical formulations containing the non-binding protein-drug conjugate s described herein can be prepared by available procedures using available ingredients. The formulations can contain pharmaceutically acceptable carriers, vehicles, and adjuvants. For example, the therapeutic agents can be formulated with common excipients, diluents, or carriers, and formed into tablets, capsules, solutions, suspensions, powders, aerosols, and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Agents for retarding dissolution can also be included such as paraffin. Resorption accelerators such as quaternary ammonium compounds can also be included. Surface active agents such as cetyl alcohol and glycerol monostearate can be included. Adsorptive pharmaceutical carriers such as kaolin and bentonite can be added. Preservatives can also be added. The compositions of the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible, for example, to prepare solutions using one or more aqueous or organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

The non-binding protein-drug conjugates described herein can be formulated for parenteral administration (e.g., by injection, for example, bolus injection or continuous infusion into the joint, a draining lymph node of the inflammatory arthritis-affected joint, a subcutaneous tissue in the vicinity of the inflammatory arthritis-affected joint, or a joint capsule of the inflammatory arthritis-affected joint) and can be presented in unit dose form in ampoules, pre-filled syringes, small volume infusion containers or in multi-dose containers.

A dose of the non-binding protein-drug conjugate can be an amount sufficient for the pharmaceutically active moiety or a diagnostic moiety to have the desired effect on the cells in the macropinocytosis-positive disease state.

Preservatives can be added to help maintain the shelve life of the dosage form. The active agents and other ingredients can form suspensions, solutions, or emulsions in oily or aqueous vehicles, and can contain formulatory agents such as suspending, stabilizing and/or dispersing agents. Alternatively, the therapeutic agents and other ingredients can be in powder form, obtained by aseptic isolation of sterile solid or by lyophilization from solution, for constitution with a suitable vehicle, e.g., sterile, pyrogen-free water, before use.

The compositions can also include antioxidants, surfactants, film-forming, keratolytic or comedolytic agents. Antioxidants such as t-butylhydroquinone, butylated hydroxyanisole, butylated hydroxytoluene and α-tocopherol and its derivatives can be added.

The compositions can include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the pharmaceutical carriers and/or diluents that are useful in the pharmaceutical formulations of the present invention include water and physiologically acceptable buffered saline solutions such as phosphate buffered saline solutions pH 7.0-8.0

Furthermore, the active ingredients can also be used in combination with other therapeutic agents, for example, pain relievers, anti-inflammatory agents, anti-cancer agents and the like, whether for the conditions described or some other condition.

### Kits

The present invention further pertains to a packaged pharmaceutical composition such as a kit or other container for detecting, controlling, preventing, or treating a disease. The kits of the invention can be designed for detecting, controlling, preventing, or treating diseases such as those described herein (e.g., an inflammatory condition). In one embodiment, the kit or container can hold the non-binding protein-drug conjugate at least partially encapsulated in a biodegradable material as well as instructions for preparing a composition that includes the non-binding protein-drug conjugate.

In another embodiment, the kit or container can hold a therapeutically effective amount of a pharmaceutical composition for treating, preventing, or controlling a disease and instructions for using the pharmaceutical composition for control of the disease. The pharmaceutical composition can include at least one type of non-binding protein-drug conjugate in a therapeutically effective amount such that the disease is controlled, prevented, or treated. Such a composition can be in liquid form, powder form or other form permitting ready administration to a patient.

The kits of the invention can also comprise containers with tools useful for administering the compositions of the invention. Such tools can include syringes, swabs, catheters, antiseptic solutions, and the like. Some kits can include all of the desired tools, solutions, compounds, including mixing vessels, utensils, and injection devices, to treat a patient according to any of the methods described herein. In one embodiment, a kit includes the non-binding protein-drug conjugate of the various embodiments described herein. The non-binding protein-drug conjugate can be sterile-packaged as a dry powder in a suitable container (e.g., a substantially water-impermeable) such as a syringe, vial (e.g., the vial can include a septum and/or a crimp seal; and the vial can optionally comprise an inert atmosphere, such as a nitrogen atmosphere or dry air) or pouch (e.g., a pouch comprising a moisture barrier; and the pouch can optionally comprise an inert atmosphere, such as a nitrogen atmosphere, or dry air). The vial containing the non-binding protein-drug conjugate can have an injection cap that does not require the use of a needle to withdraw the suspended solution can be used to avoid damaging the non-binding protein-drug conjugate or separating the particles from the solution under negative pressure. The kit can also include a desiccant. The desiccant can be included in the pouch or integrated into the layers of the pouch material. In some embodiments, the non-binding protein-drug conjugate can be sterile-packaged in frozen vehicle. As mentioned previously, the vehicle can be any suitable vehicle, including flowable vehicles (e.g., a liquid vehicle) such as a flowable, bioresorbable polymer, saline, sterile water, Ringer's solutions, and isotonic sodium chloride solutions. Examples of vehicles include, but are not limited, to Sodium Chloride Injection USP (0.9%), Ringer's Injection USP, Lactated Ringer's Injection USP, Sodium Lactate Injection USP, Dextrose Injection USP (5% or 10%), Bacteriostatic Water for Injection USP and Sterile Water for Injection USP. In some examples, the non-binding protein-drug conjugate can be suspended in water; pre-filled into a container, such as a syringe; and frozen.

The kit can include at least one static mixing element, such as a one that is attached to a syringe. In some embodiments, the user provides a static mixing element to deliver the non-binding protein-drug conjugate.

The kit can also include beads that serve to, among other things, disaggregate any non-binding protein-drug conjugate agglomeration that can occur when the non-binding protein-drug conjugate of the various embodiments described herein are reconstituted with a vehicle. In some embodiments, the beads are sufficiently larger than the non-binding protein-drug conjugate, so that the non-binding protein-drug conjugate can be selectively delivered to the injection site, while the beads remain in the injection device (e.g., a syringe). For example, the beads can have at least one dimension that is about 1 mm. The beads can be of any suitable shape, including spherical and oval in shape. The beads can also have any suitable texture. For example, the beads can have a smooth texture and/or a rough texture. The beads can also be made of any suitable material, including glass, ceramic, metal (e.g. stainless steel), polymeric (e.g. ePTFE or polypropylene), and composite materials. The beads can be included in the kit in a separate container; in the same container as the non-binding protein-drug conjugate of the various embodiments described herein; or the user can provide beads of suitable size, shape, texture, and/or materials at the point of care.

The kit can also include an injection vehicle described herein, such as sterile water or sterile saline (e.g., in the case where the target injection area is substantially hydrophobic or lipophilic) or other suitable vehicle, including a non-aqueous vehicle (e.g., a hydrophobic, liquid vehicle described herein). Prior to administration, the non-binding protein-drug conjugate can be added to the injection vehicle to form a suspension and agitated (e.g., stirred, shaken or vortexed) to maximize homogeneity. In some embodiments, the non-binding protein-drug conjugate can come in the kit, suspended in a vehicle, such as a non-aqueous vehicle (e.g., a hydrophobic, liquid vehicle described herein).

The kit can further include a hypodermic needle or other delivery device, such as a cannula, catheter, or other suitable tubing. The kit can further include instructions, dosage tables, and other pertinent information for a practitioner.

The kit can include one or more additional Active Pharmaceutical Ingredients (APIs), such as a local anesthetic, either in the same container as the non-binding protein-drug conjugate of the various embodiments described herein or in a separate container, such that the API in a separate container can be combined with the non-binding protein-drug conjugate and vehicle to provide a bolus of an API upon administration (e.g., injection) of the non-binding protein-drug conjugate. In other embodiments, the user can provide one or more additional APIs that can be combined with the non-binding protein-drug conjugate of the various embodiments described herein, at the point of care. In one specific example, a kit comprises a pre-filled syringe for non-binding protein-drug conjugate in 2 ml 1% lidocaine. The non-binding protein-drug conjugate and lidocaine are, in some embodiments, lyophilized and reconstituted with a suitable vehicle (e.g., sterile saline or water) that suspends the non-binding protein-drug conjugate and dissolves the powder prior to injection.

The kits can include instructions or printed indicia, to provide for directions for reconstituting the contents of the multiple packages, and/or for the administration of the resulting composition (e.g., the injectable compositions). For example, instructions on printed indicia can instruct injection into biological tissue including at least one of fatty tissue, epidural tissue, and at or near a targeted nerve.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The invention will be further described by the following non-limiting examples.

### Examples

### 1. Examples of Non-Binding Protein Scaffolds

### A. Affibody

Affibodies are single domain proteins that were originally derived from the B-domain in the immunoglobulin-binding region of staphylococcal protein A. The B-domain is a relatively short cysteine-free peptide of 58 amino acids that is folded into a three-helical bundle structure. The B-domain was mutated at key positions for enhanced chemical stability and the resulting engineered variant was denoted the Z-domain. The engineered Z-domain retained its affinity for the Fc part of the antibody while the weaker affinity for the Fab region was almost completely lost. Affibodies are based on the Z-domain scaffold, but with specific binding for theoretically any given target. New affibody molecules are identified by randomization of 13 amino acid positions in helices one and two that comprise the original Fc-binding surface of the Z-domain.

Affibodies can have the protein sequence shown
below (SEQ ID NO: 1):
VDNKFNKEQQNAFYEILHLPNLNEEQRNAFIQSLKDDPSQSANLLAEAKK
LNDAQAPK

The mutation of any combination of these 13 amino acid residues to produce a non-binding affibody can facilitate cellular internalization through macropinocytosis.

A non-binding sequence of an affibody can be of
the formula shown below (SEQ ID NO: 2):
VDNKFNKEX₉X₁₀X₁₁AX₁₃X₁₄EIX₁₇X₁₈LPNLNX₂₄X₂₅QX₂₇X₂₈A
FIX₃₂SLX₃₅DDPSQSANLLAEAKKLNDAQAPK

Where a combination of X₉, X₁₀, X₁₁, X₁₃, X₁₄, X₁₇, X₁₈, X₂₄, X₂₅, X₂₇, X₂₈, X₃₂, X₃₅ results in ‘non-binding’ affibody scaffold.

In one example, the non-binding sequence of an
affibody can be the sequence shown below
(SEQ ID NO: 3):
VDNKFNKESSSASSEISSLPNLNSSQSSAFISSLSDDPSQSANLLAEAKK
LNDAQAPK

### B. Affilin

Affilins are structurally derived from either ubiquitin or gamma-B crystallin. They have surface-exposed amino acids that can be mutated to form affilins with affinity for specific antigens. Ubiquitin-based affilins consist of 76 amino acids (SEQ ID NO: 4) or 162 amino acids (SEQ ID NO: 7). Extracellular ubiquitin has been reported to be an agonist of CX chemokine receptor 4. New affilin molecules are identified from the ubiquitin-based scaffold by randomization of 8 amino acid positions of the ubiquitin monomer or 15 amino acid positions of the ubiquitin dimer. Mutation of any combination of these 8 amino acid residues (SEQ ID NO: 5) or 15 amino acids (SEQ ID NO: 8) to produce a non-binding affilin can facilitate cellular internalization through macropinocytosis. Such sequences include SEQ ID NO: 6 and SEQ ID NO: 9. Gamma-crystallin-based affilins consist of 175 amino acids (SEQ ID NO: 10). Gamma-crystallin is an exclusively structural protein, with no known affinity for other molecules or any detectable enzymatic activity. New affilin molecules are identified from the gamma-crystallin-based scaffold by randomization of 8 amino acids positions. Mutation of any combination of these 8 amino acid residues (SEQ ID NO: 11) to produce a non-binding affilin can facilitate cellular internalization through macropinocytosis. One such sequence includes SEQ ID NO: 12.

Affilin Scaffold (ubiquitin) non-binding
protein scaffold can be the sequence shown below
(SEQ ID NO: 4):
MQIFVKTLTGKTITLEVEPSDTIENVKAKIQDKEGIPPDQQRLIF
AGKQLEDGRTLSDYNIQKESTLHLVLRLRGG

Affilin Scaffold (ubiquitin) non-binding
protein scaffold can be the sequence shown below
(SEQ ID NO: 5):
MX₂IX₄VX₆TLTGKTITLEVEPSDTIENVKAKIQDKEGIPPDQQR
LIFAGKQLEDGRTLSDYNIX₆₂X₆₃X₆₄X₆₅X₆₆LHLVLRLRGG

Where a combination of X₂, X₁₄, X₆, X₆₂, X₆₃, X₆₄, X₆₅, X₆₆ results in ‘non-binding’ affilin scaffold.

Affilin Scaffold (ubiquitin) non-binding
protein scaffold can be the sequence shown below
(SEQ ID NO: 6):
MSISVSTLTGKTITLEVEPSDTIENVKAKIQDKEGIPPDQQRLIF
AGKQLEDGRTLSDYNISSSSSLHLVLRLRGG
Affilin Scaffold (ubiquitin dimer) non-binding
protein scaffold can be the sequence shown below
(SEQ ID NO: 7):
MQIFVKTLTGKTITLEVEPSDTIENVKAKIQDKEGIPPDQQRLIF
AGKQLEDGRTLSDYNIQKESTLHLVLRLRGGSGGGSGGGIGMQIF
VKTLTGKTITLEVEPSDTIENVKAKIQDKEGIPPDQQRLIFAGKQ
LEDGRTLSDYNIQKESTLHLVLRLRGG
Affilin Scaffold (ubiquitin dimer) non-binding
protein scaffold can be the sequence shown below
(SEQ ID NO: 8):
MX₂IX₄VX₆TLTGKTITLEVEPSDTIENVKAKIQDKEGIPPDQQR
LIFAGKQLEDGRTLSDYNIX₆₂X₆₃X₆₄X₆₅X₆₆LHLVLRLRGGS
GGGSGGGIGMQIFVX₉₂TX₉₄TGKTITLEVEPSDTIENVKAKIQD
KEGIPPDQQRLIFAGKQLEDGRTLSDYNIX₁₄₈X₁₄₉X₁₅₀X₁₅₁
X₁₅₂LHLVLRLRGG

Where a combination of X₂, X₁₄, X₆, X₆₂, X₆₃, X₆₄, X₆₅, X₆₆, X₉₂, X₉₄, X₁₄₈, X₁₄₉, X₁₅₀, X₁₅₁, X₁₅₂ results in ‘non-binding’ affilin scaffold.

Affilin Scaffold (ubiquitin dimer) non-binding
protein scaffold can be the sequence shown below
(SEQ ID NO: 9):
MSISVSTLTGKTITLEVEPSDTIENVKAKIQDKEGIPPDQQRLIF
AGKQLEDGRTLSDYNISSSSSLHLVLRLRGGSGGGSGGGIGMQIF
VSTSTGKTITLEVEPSDTIENVKAKIQDKEGIPPDQQRLIFAGKQ
LEDGRTLSDYNISSSSSLHLVLRLRGG
Affilin Scaffold (gamma-crystallin) non-binding
protein scaffold can be the sequence shown below
(SEQ ID NO: 10):
MGKITFYEDRGFQGHCYECSSDCPNLQPYFSRCNSIRVDSGCWML
YERPNYQGHQYFLRRGDYPDYQQWMGFNDSIRSCRLIPQHTGTFR
MRIYERDDFRGQMSEITDDCPSLQDRFHLTEVHSLNVLEGSWVLY
EMPSYRGRQYLLRPGEYRRYLDWGAMNAKVGSLRRVMDFY
Affilin Scaffold (gamma-crystallin) non-binding
protein scaffold can be the sequence shown below
(SEQ ID NO: 11):
MGX₃IX₅FX-EDRGFQGHX₁₆YX₁₈CX₂₀SDCPNLQPYFSRCNSI
X₃₇VX₃₉SGCWMLYERPNYQGHQYFLRRGDYPDYQQWMGFNDSIR
SCRLIPQHTGTFRMRIYERDDFRGQMSEITDDCPSLQDRFHLTEV
HSLNVLEGSWVLYEMPSYRGRQYLLRPGEYRRYLDWGAMNAKVGS
LRRVMDFY

Where a combination of X₃, X₅, X₇, X₁₆, X₁₈, X₂₀, X₃₇, X₃₉ results in ‘non-binding’ affilin scaffold.

Affilin Scaffold (gamma-crystallin) ‘non-binding
protein scaffold can be the sequence shown below
(SEQ. ID. NO. 12):
MGSISFSEDRGFQGHSYSCSSDCPNLQPYFSRCNSISVSSGCWML
YERPNYQGHQYFLRRGDYPDYQQWMGFNDSIRSCRLIPQHTGTFR
MRIYERDDFRGQMSEITDDCPSLQDRFHLTEVHSLNVLEGSWVLY
EMPSYRGRQYLLRPGEYRRYLDWGAMNAKVGSLRRVMDFY

### C. Anticalin

Anticalins are derived from human lipocalin, which bind vitamins, hormones, or secondary metabolites. An anticalin scaffold can be comprised of a rigid beta-barrel 0 that supports four structurally hypervariable loops that facilitate its binding properties (SEQ ID NO: 13). Mutagenesis of amino acids at the hypervariable loops allows for changing the anticalin binding properties. Mutation of any combination of these amino acid residues (SEQ ID NO: 14) to produce a non-binding anticalin can facilitate cellular internalization through macropinocytosis. One such sequence includes SEQ ID NO: 15.

Anticalin non-binding protein scaffold
can be the sequence shown below
(SEQ ID NO: 13):
QDSTSDLIPAPPLSKVPLQQNFQDNQFQGKWYVVGLAGNAILRED
KDPQKMYATIYELKEDKSYNVTSVLFRKKKCDYWIRTFVPGCQPG
EFTLGNIKSYPGLTSYLVRVVSTNYNQHAMVFFKKVSQNREYFKI
TLYGRTKELTSELKENFIRFSKSLGLPENHIVFPVPIDQCIDG
Anticalin non-binding
protein scaffold can be the sequence shown below
(SEQ ID NO: 14):
QDSTSDLIPAPPLSKVPLQQNFQDNQFQGKWYVVGX₃₆AGNX₄₀X₄₁
LREDKDPX₄₉KMX₅₂ATIYELKEDKSYNVTX₆₈VX₇₀FX₇₂X₇₃
KKCX₇₇YX₇₉IX₈₁TFVPGCQPGEFTLGX₉₆IKSX₁₀₀PGX₁₀₃
TSX₁₀₆LVRVVSTNYNQHAMVFFKX₁₂₅VX₁₂₇QNREX₁₃₂FX₁₃
4ITLYGRTKELTSELKENFIRFSKSLGLPENHIVFPVPIDQCIDG

Where a combination of X₃₆, X₄₀, X₄₁, X₄₉, X₅₂, X₆₈, X₇₀, X₇₂, X₇₃, X₇₇, X₇₉, X₈₁, X₉₆, X₁₀₀, X₁₀₃, X₁₀₆, X₁₂₅, X₁₂₇, X₁₃₂, or X₁₃₄ results in ‘non-binding’ anticalin scaffold.

Anticalin Scaffold non-binding
protein scaffold can be the sequence shown below
(SEQ ID NO: 15):
QDSTSDLIPAPPLSKVPLQQNFQDNQFQGKWYVVGSAGNSSLRED
KDPSKMSATIYELKEDKSYNVTSVSFSSKKCSYSISTFVPGCQPG
EFTLGSIKSSPGSTSSLVRVVSTNYNQHAMVFFKSVSQNRESFSI
TLYGRTKELTSELKENFIRFSKSLGLPENHIVFPVPIDQCIDG

### D. Atrimer

Atrimers are derived from C-type lectin (tetranectin) which naturally bind tretranectin ligands, such as plasminogen kringle-4. An atrimer scaffold can be comprised of three identical structural units in which the C-type lectin domains (CTLD) are positioned to form a trimerizing coil-coil region which facilitates binding. Each CTLD fragment has five loop regions 6-9 residues long that provide binding specificity (SEQ ID NO: 16). Mutation of any combination of the identified amino acid residues in the monomeric CTLD of the atrimer scaffold (SEQ ID NO: 17) to produce a non-binding atrimer scaffold can facilitate cellular internalization through macropinocytosis. One such sequence of a monomeric CTLD of the atrimer scaffold includes SEQ ID NO: 18.

Monomeric CTLD of Atrimer non-binding
protein scaffold can be the sequence shown below
(SEQ ID NO: 16):
MELWGAYLLLCLFSLLTQVTTEPPTQKPKKIVNAKKDVVNTKMFE
ELKSRLDTLAQEVALLKEQQALQTVCLKGTKVHMKCFLAFTQTKT
FHEASEDCISRGGTLGTPQTGSENDALYEYLRQSVGNEAEIWLGL
NDMAAEGTWVDMTGARIAYKNWETEITAQPDGGKTENCAVLSGAA
NGWFDKRCRDQLPYICQFGIV
Monomeric CTLD of Atrimer non-binding
protein scaffold can be the sequence shown below
(SEQ ID NO: 17):
MELWGAYLLLCLFSLLTQVTTEPPTQKPKKIVNAKKDVVNTKMFE
ELKSRLDTLAQEVALLKEQQALQTVCLKGTKVHMKCFLAFTQTKT
FHEASEDCISRGGTLGTPQTGSENDALYEYLRQSVGNEAEIWLGL
NX₁₃₇X₁₃₈X₁₃₉X₁₄₀X₁₄₁X₁₄₂X₁₄₃WVDMTGARIAYKNWET
EITAQPDX₁₆₇X₁₆₈X₁₆₉X₁₇₀ENCAVLSGAANGWFDKRCRDQL
PYICQFGIV

Where a combination of X₁₃₇, X₁₃₈, X₁₃₉, X₁₄₀, X₁₄₁, X₁₄₂, X₁₄₃, X₁₆₇, X₁₆₈, X₁₆₉, or X₁₇₀ of the monomeric CTLD results in a ‘non-binding’ atrimer scaffold.

Monomeric CTLD of Atrimer non-binding
protein scaffold can be the sequence shown below
(SEQ ID NO: 18):
MELWGAYLLLCLFSLLTQVTTEPPTQKPKKIVNAKKDVVNTKMFE
ELKSRLDTLAQEVALLKEQQALQTVCLKGTKVHMKCFLAFTQTKT
FHEASEDCISRGGTLGTPQTGSENDALYEYLRQSVGNEAEIWLGL
NSSSSSSSWVDMTGARIAYKNWETEITAQPDSSSSENCAVLSGAA
NGWFDKRCRDQLPYICQFGIV

### E. DARPin

DARPins are protein scaffolds based on ankyrin repeats. Ankyrin repeats found in eukaryotic cell proteins bind to various targets, including those involved in cytoskeletal organization and regulation of enzyme activity. The various proteins containing ankyrin repeats were used to create a consensus sequence for the DARPin scaffold that contains variable regions that can facilitate target binding (SEQ ID NO: 19). Mutation of any combination of the identified amino acid residues in the DARPin scaffold (SEQ ID NO: 20) to produce a non-binding DARPin scaffold can facilitate cellular internalization through macropinocytosis. One such sequence of a DARPin scaffold includes SEQ ID NO: 21.

DARPin non-binding
protein scaffold can be the sequence shown below
(SEQ ID NO: 19):
DLGKKLLEAARAGQDDEVRILMANGADVNA{BXXGXTPLHLAAXX
GHLEIVEVLLKZGADVNAX}_{y}DKFGKTAFDISIDNGNEDLAEILQ
KLN

Where B=D, N, S, T, and X=all amino acids except G, P, and C; Z=N, H, Y, and y=at least one sequence repeat.

DARPin non-binding
protein scaffold can be the sequence shown below
(SEQ ID NO: 20):
DLGKKLLEAARAGQDDEVRILMANGADVNA{BXXGXTPLHLAAXX
GHLEIVEVLLKZGADVNAX}_{y}DKFGKTAFDISIDNGNEDLAEILQ
KLN

Where B=D, N, S, T, and X=all amino acids except G, P, and C; Z=N, H, Y, and y=at least one sequence repeat. And where a combination of B, X, and Z amino acids results in a ‘non-binding’ DARPin scaffold.

DARPin non-binding
protein scaffold can be the sequence shown below
(SEQ ID NO: 21):
DLGKKLLEAARAGQDDEVRILMANGADVNADSSGSTPLHLAASSG
HLEIVEVLLKNGADVNASDKFGKTAFDISIDNGNEDLAEILQKLN

### F. Fynomers

Fynomers are protein scaffolds derived from human Fyn SH3 domain. The Fyn SH3 domain is composed of two anti-parallel beta-sheets and contains two flexible loops that facilitate interaction with target proteins (SEQ ID NO: 22). Mutation of any combination of the identified amino acid residues in the fynomer scaffold (SEQ. ID. NO. 23) to produce a non-binding fynomer scaffold can facilitate cellular internalization through macropinocytosis. One such sequence of a fynomer scaffold includes SEQ ID NO: 24.

Fynomer non-binding protein scaffold can
be the sequence shown below
(SEQ ID NO: 22):
GVTLFVALYDYEARTEDDLSFHKGEKFQILNSSEGDWWEARSLTT
GETGYIPSNYVAPVDSIQ
Fynomer non-binding protein scaffold can
be the sequence shown below
(SEQ ID NO: 23):
GVTLFVALYDYX₁₂X₁₃X₁₄X₁₅X₁₆X₁₇DLSFHKGEKFQILX₃₁
X₃₂X₃₃X₃₄GDWWEARSLTTGETGYIPSNYVAPVDSIQ

Where a combination of X₁₂, X₁₃, X₁₄, X₁₅, X₁₆, X₁₇, X₃₁, X₃₂, X₃₃, X₃₄ results in a ‘non-binding’ fynomer scaffold.

Fynomer Scaffold non-binding
protein scaffold can be the sequence shown below
(SEQ ID NO: 24):
GVTLFVALYDYSSSSSSDLSFHKGEKFQILSSSSGDWWEARSLTT
GETGYIPSNYVAPVDSIQ

### G. Kunitz Domains

Kunitz domain scaffolds are derived from Kunitz-type protease inhibitors, such as aprotinin. Kunitz domains are disulfide rich alpha/beta folds comprised of approximately 50-60 amino acid residues.

### H. Obodies

Obodies are derived from the OB-fold, a versatile single-domain protein binding module that binds protein, carbohydrate, nucleic acid, and small-molecule ligands. It is a 5-stranded beta-barrel domain with a concave binding face (SEQ ID NO: 25). Mutation of any combination of the identified amino acid residues in the obody scaffold (SEQ. ID. NO. 26) to produce a non-binding obody scaffold can facilitate cellular internalization through macropinocytosis. One such sequence of an obody scaffold includes SEQ ID NO: 27.

Obody non-binding protein scaffold can
be the sequence shown below
(SEQ ID NO: 25):
VYPKKTHWTAEITPNLHGTEVVVAGWVWELRDIGRVKFVVVRDRE
GGAFVQVTLKAGKTPDHLFKVFAELSREDVVVIKGIVEASKIAKS
GVEIFPSEIWILNKAKPLPID
Obody non-binding protein scaffold can
be the sequence shown below
(SEQ ID NO: 26):
VYPKKTHWTAEITPNLHGTEVVVAGWVX₂₈X₂₉LX₃₁DX₃₃GX₃₅
X₃₆KX₃₈VX₄₀VX₄₂DREGGAX₄₉VX₅₁VX₅₃LX₅₅AGKTPDHLF
KVFAELSREDVVVIKGIVEASKX₈₇X₈₈X₈₉X₉₀GVEIFPSEIWI
LNKAKPLPID

Where a combination of X₂₈, X₂₉, X₃₁, X₃₃, X₃₅, X₃₆, X₃₈, X₄₀, X₄₂, X₄₉, X₅₁, X₅₃, X₅₅, X₈₇, X₈₈, X₈₉, X₉₀ results in a ‘non-binding’ obody scaffold.

Obody non-binding protein scaffold can be the
sequence shown below (SEQ ID NO: 27):
VYPKKTHWTAEITPNLHGTEVVVAGWVSSLSDSGSSKSVSVSDREGGASV
SVSLSAGKTPDHLFKVFAELSREDVVVIKGIVEASKSSSSGVEIFPSEIW
ILNKAKPLPID

### I. Alphabody

Alphabodies are computationally designed protein scaffolds and do not have a counterpart in nature. They are composed of a single contiguous polypeptide chain designed to adopt a triple-helix coiled-coil fold. Three alpha-helices composed of four heptad repeats are connected with two linkers (SEQ ID NO: 28). Alphabody libraries can be generated by randomization of specific residues in the alpha-helices. Mutation of any combination of the identified amino acid residues in the alphabody scaffold (SEQ. ID. NO. 29, SEQ ID NO: 30, SEQ ID NO: 31) to produce a non-binding alphabody scaffold can facilitate cellular internalization through macropinocytosis. Such sequences of an alphabody scaffold include SEQ ID NO: 32, SEQ ID NO: 33, and SEQ ID NO: 34.

Alphabody non-binding protein scaffold can be
the sequence shown below (SEQ ID NO: 28):
IEEIQKQIAAIQKQIAAIQKQIYRMTGGSGGGSGGGSGGGSGMSIEEIQK
QIAAIQKQIAAIQKQIYRMTTGGSGGGSGGGSGGGSGMSIEEIQKQIAAI
QKQIAAIQKQIYRM
Alphabody non-binding protein scaffold 1 can be
the sequence shown below (SEQ ID NO: 29):
IEEIQKX₇IAX₁₀IQEX₁₄IAX₁₇IQKX₂₁IYX₂₄MTGGSGGGSGGGSGG
GSGMSIEEIQKQIAAIQKQIAAIQKQIYRMTTGGSGGGSGGGSGGGSGMS
IEEIQKQIX₉₈AIX101EQIX₁₀₅AIX₁₀₈KQIX₁₁₂AM

Where a combination of X₇, X₁₀, X₁₄, X₁₇, X₂₁, X₂₄, X₉₈, X₁₀₁, X₁₀₅, X₁₀₈, X₁₁₂ results in a ‘non-binding’ alphabody scaffold.

Alphabody non-binding protein scaffold 2 can be
the sequence shown below (SEQ ID NO: 30):
IEEIQKQIAAIQKQIAAIQKQIYAMTGGSGGGSGGGSGGGSGMSIEEIQK
QIAAIQKQIAAIQKQIYRMTTGGSGGGSGGGSGGGSGMSIEEIQX₉₅QI
X₉₈X₉₉IQX₁₀₂QIX₁₀₅X₁₀₆IQX₁₀₉QIX₁₁₂X₁₁₃M

Where a combination of X₉₅, X₉₈, X₉₉, X₁₀₂, X₁₀₅, X₁₀₆, X₁₀₉, X₁₁₂, X₁₁₃ results in a ‘non-binding’ alphabody scaffold.

Alphabody non-binding protein scaffold 3 can be
the sequence shown below (SEQ ID NO: 31):
IQQIQKX₇IAX₁₀IQEX₁₄IYX₁₇MTGGSGGGSGGGSGGGSGMDIQQIQK
QIAAIQKQIYAMTTGGSGGGSGGGSGGGSGMDIQQIQKQIX₈₃AIX₈₆EQ
IX₉₀AM

Where a combination of X₇, X₁₀, X₁₄, X₁₇, X₈₃, X₈₆, X₉₀ results in a ‘non-binding’ alphabody scaffold.

Alphabody non-binding protein scaffold can be
the sequence shown below (SEQ ID NO: 32):
IEEIQKSIASIQESIASIQKSIYSMTGGSGGGSGGGSGGGSGMSIEEIQK
QIAAIQKQIAAIQKQIYRMTTGGSGGGSGGGSGGGSGMSIEEIQKQISAI
SEQISAISKQISAM
Alphabody non-binding protein scaffold can be
the sequence shown below (SEQ ID NO: 33):
IEEIQKQIAAIQKQIAAIQKQIYAMTGGSGGGSGGGSGGGSGMSIEEIQK
QIAAIQKQIAAIQKQIYRMTTGGSGGGSGGGSGGGSGMSIEEIQSQISSI
QSQISSIQSQISSM
Alphabody non-binding protein scaffold can be
the sequence shown below (SEQ ID NO: 34):
IQQIQKSIASIQESIYSMTGGSGGGSGGGSGGGSGMDIQQIQKQIAAIQK
QIYAMTTGGSGGGSGGGSGGGSGMDIQQIQKQISAISEQISAM

### J. Nanofitin/Affitin Scaffold

The nanofitin scaffold, also known as affitin, is derived from the DNA binding Sac7d family and homologous OB-fold proteins. New binders can be derived from randomization of the amino acids on the binding surface of Sac7d (SEQ ID NO: 35). Mutation of any combination of the identified amino acid residues in the nanofitin scaffold (SEQ. ID. NO. 36) to produce a non-binding nanofitin scaffold can facilitate cellular internalization through macropinocytosis. One such sequence of a nanofitin scaffold includes SEQ ID NO: 37.

Nanofitin/Affitin non-binding protein scaffold
can be the sequence shown below (SEQ ID NO: 35):
MVKVKFKYKGEEKEVDTSKIKKVWRVGKMVSFTYDDNGKTGRGAVSEKDA
PKELLDMLARAEREKK
Nanofitin/Affitin non-binding protein scaffold
can be the sequence shown below (SEQ ID NO: 36):
MVKVKFX₇X₈X₉GEEKEVDTSKIX₂₁X₂₂VX₂₄RX₂₆GKX₂₉VX₃₁FX₃₃
YDDNGKX₄₀GX₄₂GX₄₄VX₄₆EKDAPKELLDMLARAEREKK

Where a combination of X₇, X₈, X₉, X₂₁, X₂₂, X₂₄, X₂₆, X₂₉, X₃₁, X₃₃, X₄₀, X₄₂, X₄₄, X₄₆ results in a ‘non-binding’ nanofitin scaffold.

Nanofitin/Affitin non-binding protein scaffold
can be the sequence shown below (SEQ ID NO: 37):
MVKVKFSSSGEEKEVDTSKISSVSRSGKSVSFSYDDNGKSGSGSVSEKDA
PKELLDMLARAEREKK

### K. Affimer/Adhiron Scaffold

Affimers, derived from adhiron scaffolds, are based on a consensus sequence of plant-derived phytocystatins, which are small (approximately 100 amino acids) protein inhibitors of cysteine proteases (SEQ ID NO: 38). Randomization of nine amino acids in each of the two binding loops of the sequence can generate affimers with binding capabilities. Mutation of any combination of the identified amino acid residues in the affimer scaffold (SEQ. ID. NO. 39) to produce a non-binding affimer scaffold can facilitate cellular internalization through macropinocytosis. One such sequence of an affimer scaffold includes SEQ ID NO: 40.

Affimer/Adhiron non-binding protein scaffold
can be the sequence shown below (SEQ ID NO: 38)
VRAVPGNENSLEIEELARFAVDEHNKKENALLEFVRVVKAKEQVVAGTMY
YLTLEAKDGGKKKLYEAKVMVKPWENFKELQEFKPVGDA
Affimer/Adhiron non-binding protein scaffold
can be the sequence shown below (SEQ ID NO: 39):
VRAVPGNENSLEIEELARFAVDEHNKKENALLEFVRVVKAKEQX₄₄X₄₅
X₄₆X₄₇X₄₈X₄₉X₅₀X₅₁X₅₂TMYYLTLEAKDGGKKKLYEAKVMVKX₇₈
X₇₉X₈₀X₈₁X₈₂X₈₃X₈₄X₈₅X₈₆NFKELQEFKPVGDA

Where a combination of X₄₄, X₄₅, X₄₆, X₄₇, X₄₈, X₄₉, X₅₀, X₅₁, X₅₂, X₇₈, X₇₉, X₈₀, X₈₁, X₈₂, X₈₃, X₈₄, X₈₅, X₈₆ results in a ‘non-binding’ affimer scaffold.

Affimer/Adhiron non-binding protein scaffold
can be the sequence shown below (SEQ ID NO: 40):
VRAVPGNENSLEIEELARFAVDEHNKKENALLEFVRVVKAKEQSSSSSSS
SSTMYYLTLEAKDGGKKKLYEAKVMVKSSSSSSSSSNFKELQEFKPVGDA

### L. Repebody Scaffold

A repebody scaffold was computationally designed based on variable lymphocyte receptors, which are nonimmunoglobulin antibodies composed of Leucine-rich repeat modules from jawless vertebrates. Randomization of amino acids at two adjoining repeat modules of the repebody scaffold at three hypervariable sites (positions 8, 10, and 11) allows for the generation of target-binding repebodies. Randomization of these hypervariable regions in the repebody to produce a non-binding repebody scaffold can facilitate cellular internalization through macropinocytosis.

### Example 2: Cytotoxicity of Non-Binding Protein-Drug Conjugates

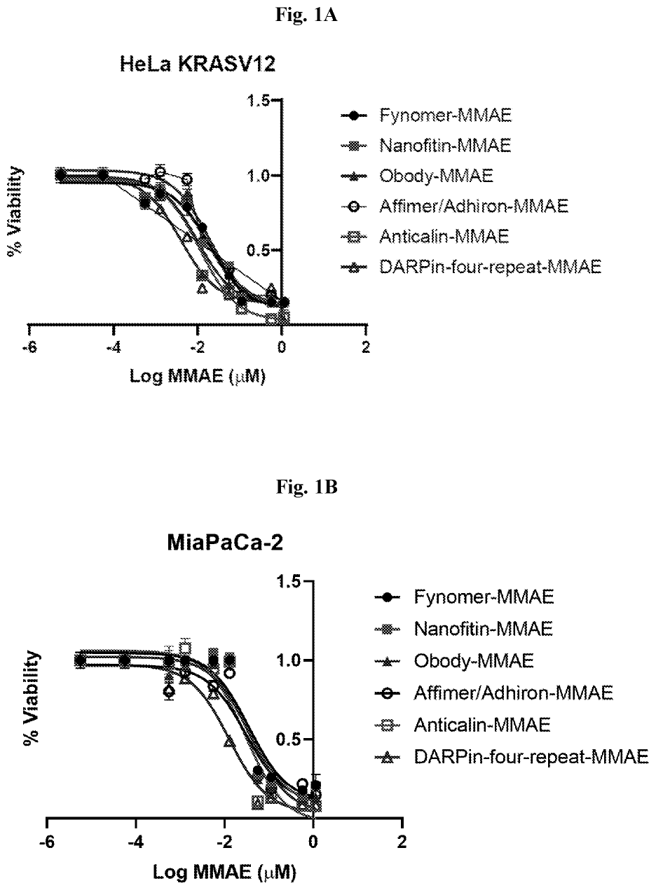

Six non-binding protein-drug conjugates were constructed with the drug monomethyl auristatin (MMAE), a cytotoxic chemotherapy agent, and their cytotoxicity evaluated (FIGS. 1A-1B). MMAE comprises a VC linker conjugated to a C-terminal cysteine on each non-binding protein scaffold to form non-binding protein-MMAE conjugates.

HeLa cells expressing KRasV12 (referred to as HeLa KRasV12) and a human pancreatic cancer cells MIA-PaCa-2 were plated in 96 well plates in Dulbecco's Modified Eagle Medium (DMEM) complete growth medium containing 10 Fetal Bovine Serum (FBS). 25,000 cells were plated per well. 24 hours after plating, cells were treated with vehicle control or non-binding protein-MMAE conjugate. Nine serial dilutions were tested for each non-binding protein-MMAE conjugate. On day 5 post-treatment, viability was assessed by Syto60 red fluorescent nucleic acid staining that exhibits bright, red fluorescence upon binding to nucleic acids. The assay was performed three independent times in biological triplicate.

The six non-binding protein scaffolds used to construct the six non-binding protein-MMAE conjugates depicted in FIGS. 1A-1B are as follows:

### 1. Fynomer-MMAE:

The fynomer has the following sequence
(SEQ ID NO: 24):
GVTLFVALYDYSSSSSSDLSFHKGEKFQILSSSSGDWWEARSLTTGETGY
IPSNYVAPVDSIQ

### 2. Nanofitin-MMAE

The nanofitin has the following sequence
(SEQ ID NO: 37):
MVKVKFSSSGEEKEVDTSKISSVSRSGKSVSFSYDDNGKSGSGSVSEKDA
PKELLDMLARAEREKK

### 3. Obody-MMAE

The obody has the following sequence
(SEQ ID NO: 27):
VYPKKTHWTAEITPNLHGTEVVVAGWVSSLSDSGSSKSVSVSDREGGASV
SVSLSAGKTPDHLFKVFAELSREDVVVIKGIVEASKSSSSGVEIFPSEIW
ILNKAKPLPID

### 4. Affimer/Adhiron-MMAE

The affimer/adhiron has the following sequence
(SEQ ID NO: 40):
VRAVPGNENSLEIEELARFAVDEHNKKENALLEFVRVVKAKEQSSSSSSS
SSTMYYLTLEAKDGGKKKLYEAKVMVKSSSSSSSSSNFKELQEFKPVGDA

### 5. Anticalin-MMAE

The anticalin has the following sequence
(SEQ ID NO: 15):
QDSTSDLIPAPPLSKVPLQQNFQDNQFQGKWYVVGSAGNSSLREDKDPSK
MSATIYELKEDKSYNVTSVSFSSKKCSYSISTFVPGCQPGEFTLGSIKSS
PGSTSSLVRVVSTNYNQHAMVFFKSVSQNRESFSITLYGRTKELTSELKE
NFIRFSKSLGLPENHIVFPVPIDQCIDG

### 6. DARPin-Four-Repeat-MMAE

The DARPin-four-repeat has the following amino
acid sequence comprising SEQ ID NO: 21 with
four repeats as described in SEQ ID NO: 20,
shown below as SEQ ID NO: 41:
DLGKKLLEAARAGQDDEVRILMANGADVN(ADSSGSTPLHLAASSGHLEI
VEVLLKNGADVNAS)₄DKFGKTAFDISIDNGNEDLAEILQKLN

The non-binding protein-MMAE conjugates are cell impermeable complexes that enter cells through macropinocytosis selective cellular uptake pathways. Cytotoxicity of each of the above six non-binding protein-MMAE conjugate were determined by treating HeLa KRasV12 (FIG. 1A) and MiaPaCa-2 (FIG. 1B) cells with increasing concentrations of the non-binding protein-MMAE conjugates. As shown, dose-dependent cytotoxicity occurred with all six non-binding protein-MMAE conjugates.

### Example 3

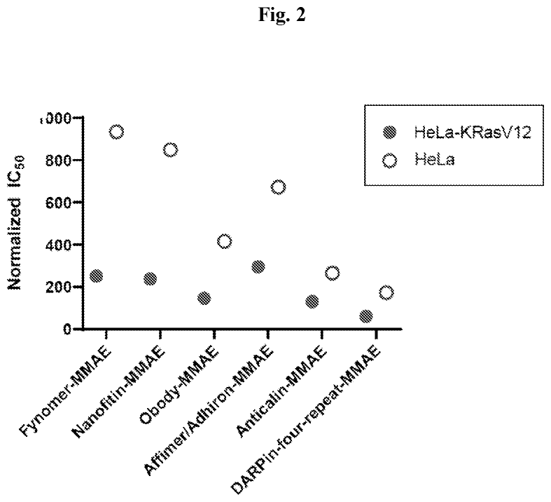

To determine if Ras mutational status confers differential cytotoxicity, the IC₅₀ values of the six non-binding protein-MMAE conjugates from Example 2 were determined in HeLa and HeLa KRasV12 cells (FIG. 2). HeLa cells and HeLa cells expressing KRasV12 were plated in 96 well plates in DMEM complete growth medium containing 10% FBS. 25,000 cells were plated per well. 24 hours after plating, cells were treated with vehicle control, free MMAE, or non-binding protein-MMAE conjugate. Nine serial dilutions were tested for free MMAE and each non-binding protein-MMAE conjugate. On day 5 post-treatment, viability of the cells was assessed by Syto60 staining. IC₅₀ of non-binding protein-MMAE conjugates was normalized to IC₅₀ of free MMAE to control for variability of sensitivity to MMAE between HeLa and HeLa KRasV12 cells. The assay was performed three independent times in biological triplicate.

After normalizing the IC₅₀, a significant decrease in cytotoxicity with each non-binding protein-MMAE conjugate was observed in the HeLa cell line (macropinocytosis-negative) compared to the HeLa KRasV12 cell line (macropinocytosis-positive) (FIG. 2, Table 1 below), suggesting cellular uptake of the non-binding protein-MMAE conjugates is macropinocytosis dependent.

| Non-Binding | IC₅₀ Fold Change |
|---|---|
| Protein-MMAE | (HeLa/HeLa- |
| Conjugate | KRasV12) |
| Fynomer | 3.7 |
| Nanofitin | 3.5 |
| Obody | 2.8 |
| Affimer/Adhiron | 2.3 |
| Anticalin | 2 |
| DARPin-Four- | 2.9 |
| Repeat | |

### Statements

The following statements are intended to describe and summarize various embodiments of the technology according to the foregoing description in the specification.
1. A pharmaceutical composition comprising:
   a non-binding protein-drug conjugate having increased susceptibility to macropinocytosis by a population of cells in a
   macropinocytosis-
      positive disease state relative to a population of cells that are not in a macropinocytosis-positive disease state, the non-binding protein-drug conjugate comprising:
         a first portion comprising a non-binding protein scaffold
         that does not substantially bind to a cell surface, wherein the non-binding protein scaffold does not comprise a non-binding fibronectin type III (FN3) domain;
   a peptide linker coupled to the first portion; and
   a second portion coupled to the peptide linker, wherein the second portion comprises a pharmaceutically active moiety or a diagnostic moiety.
2. The composition of statement 1, wherein the cell in the macropinocytosis-positive disease state is a cancer cell characterized by increased macropinocytosis relative to a noncancer cell.
3. The composition of statement 1, wherein the macropinocytosis-positive disease state is a neurodegenerative disease, an infectious disease, an inflammatory disease, or a bone disease.
4. The composition of statement 1, wherein the non-binding protein scaffold comprises one or more amino acid substitutions in a protein binding sequence of a native non-antibody protein scaffold amino acid sequence.
5. The composition of statement 1, wherein the non-binding protein scaffold comprises one or more amino acid substitutions in a protein binding sequence of an antibody-based protein scaffold.
6. The composition of statement 5, wherein the antibody-based protein scaffold is selected from an immunoglobulin, Fab, ScFv, Abdurin, Nanobody, or Humabody.
7. The composition of statement 1, wherein non-binding protein scaffold is more than 5 kDa to increase susceptibility of the non-binding protein scaffold to being engulfed through macropinocytosis by the cell in the macropinocytosis-positive disease state.
8. The composition of any one of statement 1-4, wherein the first portion comprises an amino acid sequence of SEQ ID NOS: 1-41.
9. The composition of any one of statement 1-4, wherein the peptide linker is a cleavable linker.
10. The composition of any one of statement 1-4, wherein the peptide linker is a non-cleavable linker.
11. The composition of any one of statement 1-4, further comprising a pharmaceutically acceptable carrier.
12. The composition of any one of statement 1-4, wherein the pharmaceutically active moiety is a cancer therapeutic.
13. The composition of statement 12, wherein the cancer therapeutic comprises an antimetabolite, an alkaloid, an alkylating agent, an anti-mitotic agent, an antitumor antibiotic, a DNA binding drug, a toxin, an antiproliferative drug, a DNA antagonist, a radionuclide, a thermoablative agent a proteolysis targeting chimera (PROTAC), a nucleic acid inhibitor, or an immune-modulatory agent.
14. The composition of statement 13, wherein the alkaloid comprises duocarmycin, docetaxel, etoposide, irinotecan, paclitaxel, teniposide, topotecan, vinblastine, vincristine, vindesine, and analogs and derivatives thereof.
15. The composition of statement 13, wherein the alkylating agent comprises busulfan, improsulfan, piposulfan, benzodepa, carboquone, meturedepa, uredepa, altretamine, triethylenemelamine, triethylenephosphoramide, triethylenethiophosphorarnide, chlorambucil, chloranaphazine, cyclophosphamide, estramustine, ifosfamide, mechlorethamine, mechlorethamine oxide HCl, melphalan, novemebichin, perfosfamide phenesterine, prednimustine, trofosfamide, uracil mustard, carmustine, chlorozotocin, fotemustine, lomustine, nimustine, semustine ranimustine, dacarbazine, mannomustine, mitobronitol, mitolactol, pipobroman, temozolomide, and analogs and derivatives thereof.
16. The composition of statement 13, wherein the antitumor antibiotic comprises aclacinomycin, actinomycin, anthramycin, azaserine, bleomycin, cactinomycin, calicheamicin, carubicin, carzinophilin, cromomycin, dactinomycin, daunorubicin, 6-diazo-5-oxo-1-norleucine, doxorubicin, epirabicin, idarubicin, menogaril, mitomycin, mycophenolic acid, nogalamycine, olivomycin, peplomycin, pirarubicin, plicamycin, porfiromycin, puromycine, pyrrolobenzodiazepine, streptonigrin, streptozocin, tubercidin, zinostatin, zorubicin, and analogs and derivatives thereof.
17. The composition of statement 13, wherein the antimetabolite comprises SN-38, denopterin, edatrexate, mercaptopurine (6-MP), methotrexate, piritrexim, pteropterin, pentostatin (2′-DCF), tomudex, trimetrexate, cladridine, fludarabine, thiamiprine, ancitabine, azacitidine, 6-azauridine, carmofur, cytarabine, doxifluridine, emitefur, floxuridine, fluorouracil, gemcitabine, tegafur, hydroxyurea, urethane, and analogs and derivatives thereof.
18. The composition of statement 13, wherein the anti-proliferative drug comprises aceglatone, amsacrine, bisantrene, camptothecin, defosfamide, demecolcine, diaziquone, diflomotecan, eflornithine, elliptinium acetate, etoglucid, etopside, fenretinide, gallium nitrate, hydroxyurea, lamellarin D, lonidamine, miltefosine, mitoguazone, mitoxantrone, mopidamol, nitracrine, pentostatin, phenamet, podophillinic acid 2-ethyl-hydrazide, procarbazine, razoxane, sobuzoxane, spirogermanium, teniposide, tenuazonic acid, triaziquone 2,2′,2″-trichlorotriethylamine, and analogs and derivatives thereof.
19. The composition of statement 13, wherein the antimitotic agent comprises auristatin, a maytansinoid, a dolastatin, a tubulysin, a taxane, a epothilone, a vinca alkaloid, and analogs and derivatives thereof.
20. The composition of statement 13, wherein the immunomodulatory agent comprises a macrophage type-1 stimulating agent.
21. The composition of statement 20, wherein the macrophage type-1 stimulating agent comprises paclitaxel, a colony stimulating factor-1 (CSF-1) receptor antagonist, an IL-10 receptor antagonist, a Toll-like receptor (TLR)-2 agonist, a TLR-3 agonist, a TLR-4 agonist, a TLR-7 agonist, a TLR-8 agonist, and a TLR-9 agonist.
22. The composition of statement 13, wherein the immunomodulatory agent is a macrophage type-2 stimulating agent.
23. The composition of statement 22, wherein the macrophage type-2 stimulating agent comprises IL-33, IL-4 receptor agonists, glucocorticoids, IL-10 receptor agonist, IL-1 receptor agonist.
24. The composition of statement 13, wherein the immunomodulatory agent comprises a T cell stimulating agent.
25. The composition of statement 24, wherein the T cell stimulating agent is a stimulator of interferon genes (STING) agonist.
26. The composition of statement 13, wherein the immunomodulatory agent is a dendritic cell stimulating agent.
27. The composition of statement 26, wherein the dendritic cell stimulating agent comprises a CpG oligonucleotide, imiquimod, camptothecin, colchicine, podophyllotoxin, and derivatives thereof.
28. The composition of statement 13, wherein the immunomodulatory agent is a neutrophil stimulating agent.
29. The composition of statement 28, wherein the neutrophil stimulating agent comprises a recombinant granulocyte colony stimulating factor protein (filgrastim) or a pegylated recombinant granulocyte colony stimulating factor protein.
30. The composition of statement 1, wherein the pharmaceutically active moiety is an oligonucleotide.
31. The composition of statement 30, wherein the oligonucleotide comprises an siRNA, an aptamer, an miRNA, an immunostimulatory oligonucleotide, a splice-switching oligonucleotide, and guide RNA.
32. The composition of statement 1, wherein the pharmaceutically active moiety is a wound healing agent.
33. The composition of any one of statement 1-4, wherein the diagnostic moiety comprises a fluorescent dye, a radioisotope, a contrast agent suitable for imaging, a radionucleotide with a chelator, and a photosensitizer.
34. The composition of any one of statement 1-4, wherein the peptide linker comprises a C-terminal cysteine residue.
35. The composition of statement 34, wherein the peptide linker comprises one or more serine residues N-terminal to the cysteine residue.
36. The composition of statement 1-4, wherein the peptide linker has the sequence from N-terminus to C-terminus of serine-serine-serine-serine-cysteine.
37. The composition of any one of statement 1-4, wherein the second portion is bound to the C-terminal cysteine residue of the peptide linker.

38. A method of treating cancer in a subject, the method comprising:
administering to the subject a composition of any one of claims 1-37 in an
amount effective to treat the cancer.
39. The method of statement 38, wherein the cancerous cells have an oncogenic mutation in H-ras, N-ras, or K-ras genes.

The specific compositions and methods described herein are representative, exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72 (b) to allow the reader to quickly ascertain the nature and gist of the technical disclosure. The Abstract is submitted with the understanding that it will not be used to interpret or limit the scope or meaning of the claims.

1. A pharmaceutical composition comprising:
a non-binding protein-drug conjugate having increased susceptibility to macropinocytosis by a population of cells in a macropinocytosis-positive disease state relative to a population of cells that are not in a macropinocytosis-positive disease state, the non-binding protein-drug conjugate comprising:
a first portion comprising a non-binding protein scaffold that does not substantially bind to a cell surface,
wherein the non-binding protein scaffold does not comprise a non-binding fibronectin type III (FN3) domain;
a peptide linker coupled to the first portion; and
a second portion coupled to the peptide linker, wherein the second portion comprises a pharmaceutically active moiety or a diagnostic moiety.

2. The composition of claim 1, wherein the cell in the macropinocytosis-positive disease state is a cancer cell characterized by increased macropinocytosis relative to a noncancer cell.

3. The composition of claim 1, wherein the macropinocytosis-positive disease state is a neurodegenerative disease, an infectious disease, an inflammatory disease, or a bone disease.

4. The composition of claim 1, wherein the non-binding protein scaffold comprises one or more amino acid substitutions in a protein binding sequence of a native non-antibody protein scaffold amino acid sequence.

5. The composition of claim 1, wherein the non-binding protein scaffold comprises one or more amino acid substitutions in a protein binding sequence of an antibody-based protein scaffold.

6. The composition of claim 5, wherein the antibody-based protein scaffold is selected from an immunoglobulin, Fab, ScFv, Abdurin, Nanobody, or Humabody.

7. The composition of claim 1, wherein non-binding protein scaffold is more than 5 kDa to increase susceptibility of the non-binding protein scaffold to being engulfed through macropinocytosis by the cell in the macropinocytosis-positive disease state.

8. The composition of claim 1, wherein the first portion comprises an amino acid sequence of SEQ ID NOS: 1-41.

9. The composition of claim 1, wherein the peptide linker is a cleavable linker.

10. The composition of claim 1, wherein the peptide linker is a non-cleavable linker.

11. The composition of claim 1, further comprising a pharmaceutically acceptable carrier.

12. The composition of claim 1, wherein the pharmaceutically active moiety is a cancer therapeutic.

13. The composition of claim 12, wherein the cancer therapeutic comprises an antimetabolite, an alkaloid, an alkylating agent, an anti-mitotic agent, an antitumor antibiotic, a DNA binding drug, a toxin, an antiproliferative drug, a DNA antagonist, a radionuclide, a thermoablative agent a proteolysis targeting chimera (PROTAC), a nucleic acid inhibitor, or an immune-modulatory agent.

14-29. (canceled)

30. The composition of claim 1, wherein the pharmaceutically active moiety is an oligonucleotide.

31. (canceled)

32. The composition of claim 1, wherein the pharmaceutically active moiety is a wound healing agent.

33. The composition of claim 1, wherein the diagnostic moiety comprises a fluorescent dye, a radioisotope, a contrast agent suitable for imaging, a radionucleotide with a chelator, and a photosensitizer.

34. The composition of claim 1, wherein the peptide linker comprises a C-terminal cysteine residue.

35-36. (canceled)

37. The composition of claim 34, wherein the second portion is bound to the C-terminal cysteine residue of the peptide linker.

38. A method of treating cancer in a subject, the method comprising: administering to the subject a composition of claim 1, in an amount effective to treat the cancer.

39. The method of claim 38, wherein the cancerous cells have an oncogenic mutation in H-ras, N-ras, or K-ras genes.
